SEQUENCE LISTING (9 sequences)

SEQ ID NO: 1 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 169 aa, Mus musculus
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 3 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 4 - protein, 154 aa, Mus musculus
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 5 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - protein, 162 aa, Mus musculus
MKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 7 - DNA, 20 bp, synthetic construct
  source 1..20: mol_type = other DNA
ctcaccagattcccgaaggt

SEQ ID NO: 8 - DNA, 20 bp, synthetic construct
  source 1..20: mol_type = other DNA
cacctacctaagaaccatcc

SEQ ID NO: 9 - DNA, 20 bp, synthetic construct
  source 1..20: mol_type = other DNA
aaatatatcaccactattgc

(19) United States
(12) Patent Application Publication
(10) Pub. No.: US 2025/0017966 A1
(43) Pub. Date: Jan. 16, 2025

(54) LARGE-SCALE EXPANSION OF ENGINEERED HUMAN GAMMA DELTA T CELLS

(71) Applicant: REGENTS OF THE UNIVERSITY OF MINNESOTA, Minneapolis, MN (US)

(72) Inventors: Beau R. WEBBER, Minneapolis, MN (US); Branden S. MORIARITY, Minneapolis, MN (US); Jacob E. BRIDGE, Minneapolis, MN (US)

(21) Appl. No.: 18/711,849

(22) PCT Filed: Nov. 18, 2022
(86) PCT No.: PCT/US2022/080169

§ 371 Date: May 20, 2024

Related U.S. Application Data
(60) Provisional application No. 63/280,917, filed on Nov. 18, 2021.

(51) Int. Cl. A61K 35/17; A61K 39/00; A61P 35/00; C12N 5/0783

(52) U.S. Cl. CPC A61K 35/17; A61K 39/4611; A61K 39/4631; A61K 39/464412; A61P 35/00; C12N 5/0636; C12N 2501/2302; C12N 2501/2307; C12N 2501/2315; C12N 2501/51; C12N 2501/515; C12N 2510/00

(57) ABSTRACT

Disclosed herein are methods of stimulating and expanding polyclonal gamma delta T cells (GDTCs) in vitro. More specifically, anti-gamma delta T cell receptor (GDTCR) antibody is used in combination with anti-CD28 antibody and one or more of IL-2, IL-7, and IL-15 to efficiently expand GDTCs in vitro.

### CROSS REFERENCE TO RELATED APPLICATIONS

The present application claims priority to U.S. Provisional Patent Application No. 63/280,917 that was filed Nov. 18, 2021, the entire contents of which are hereby incorporated by reference.

### REFERENCE TO A SEQUENCE LISTING

A Sequence Listing accompanies this application and is submitted as an xml file of the sequence listing named “920171_00497.xml” which is 9,829 bytes in size and was created on Nov. 18, 2022. The sequence listing is electronically submitted via Patent Center and is incorporated herein by reference in its entirety.

### FIELD OF THE INVENTION

The invention relates to methods for the large-scale expansion of polyclonal gamma delta T cells.

### BACKGROUND

Gamma delta T cells (GDTCs) represent an emerging cell type of interest for cancer immunotherapy. Their inherent ability to target and destroy cancer cells via recognition of non-peptide antigens independent of MHC is highly advantageous and allows for allogeneic transfer irrespective of HLA match. However, their relative infrequency in peripheral blood and inefficient methods for expansion of GDTCs bearing polyclonal GDTC receptor repertoires has limited their clinical application. Exposure to aminobisphosphonates such as zoledronate has been used to expand the Vg9Vd2 subset but requires culture in the presence of other mononuclear cells (MNCs) in order for efficient activation, which is not conducive to genetic modification techniques. Further, since zoledronate expansion only grows out a single subset of GDTCs the ultimate efficacy against a broad range of malignancies is limited. Engineered feeder cell lines have also been used to expand GDTCs in vitro but suffer from poor standardization and challenges associated with clinical use.

### SUMMARY

In an aspect of the current disclosure, methods of expanding polyclonal gammadelta T cells (GDTCs) in vitro are provided. In some embodiments, the methods comprise: a) incubating GDTCs in media comprising: i) anti-gammadelta T cell receptor (GDTCR) antibody; ii) anti-CD28 antibody; and iii) one or more of IL-2, IL-7, IL-15 to activate the GDTCs; b) culturing the GDTCs to expand the GDTCs; and c) re-stimulating the GDTCs of step (b) by incubating the GDTCs in medium comprising: i) anti-gammadelta T cell receptor antibody; and ii) anti-CD28 antibody to re-stimulate the polyclonal GDTCs. In some embodiments, the GDTCs are not cultured in the presence of a bisphosphonate or in the presence of feeder cells. In some embodiments, the methods further comprising at step (b) genetically engineering the cells to alter the expression of one or more proteins in the GDTCs. In some embodiments, the cells are genetically engineered using a CRISPR/Cas system. In some embodiments, a Cas nuclease and one or more guide RNAs are introduced into the cells to allow for genetic engineering. In some embodiments, the guide RNAs are specific for at least one of cytokine inducible SH2 containing protein (CISH), programmed death-1 (PD-1), and Fas receptor (FasR) and result in reduced activity of the protein. In some embodiments, the genetic engineering comprises contacting the cells with a vector comprising one or more exogenous nucleic acids operably linked to a promoter. In some embodiments, the one or more exogenous nucleic acids encode a Cas nuclease and/or one or more guide RNAs. In some embodiments, the one or more guide RNAs are targeted to one or more genes encoding a protein in the cells. In some embodiments, the one or more guide RNAs target one or more genes encoding a protein in the cells selected from: cytokine inducible SH2 containing protein (CISH), programmed death-1 (PD-1), and Fas receptor (FasR). In some embodiments, the cells are genetically engineered by contact with a vector encoding a transposon. In some embodiments, the transposon is a TcBuster™ transposon. In some embodiments, the population of polyclonal GDTCs are expanded at least 100-fold. In some embodiments, the population of polyclonal GDTCs are expanded at least 1000-fold. In some embodiments, the population of polyclonal GDTCs are expanded at least 3000-fold. In some embodiments, the anti-gammadelta T cell receptor antibody is linked to a solid support. In some embodiments, the solid support comprises the surface of the vessel in which the GDTCs are cultured in step b). In some embodiments, the solid support comprises beads. In some embodiments, the method further comprises contacting the cells with a FasL blocking reagent. In some embodiments, step (a) to activate the GDTCs is about 1-3 days. In some embodiments, step (c) to re-stimulate the GDTCs is about 1-3 days. In some embodiments, step (b) to expand the GDTCs is greater than about 10 days, optionally about 11 days.

In another aspect of the current disclosure, gammadelta T cells (GDTCs) are provided. In some embodiments, the GDTCs are made by methods comprising: a) incubating GDTCs in media comprising: i) anti-gammadelta T cell receptor (GDTCR) antibody; ii) anti-CD28 antibody; and iii) one or more of IL-2, IL-7, IL-15 to activate the GDTCs; b) culturing the GDTCs to expand the GDTCs; and c) re-stimulating the GDTCs of step (b) by incubating the GDTCs in medium comprising: i) anti-gammadelta T cell receptor antibody; and ii) anti-CD28 antibody to re-stimulate the polyclonal GDTCs. In some embodiments, the GDTCs are not cultured in the presence of a bisphosphonate or in the presence of feeder cells. In some embodiments, the methods further comprising at step (b) genetically engineering the cells to alter the expression of one or more proteins in the GDTCs. In some embodiments, the cells are genetically engineered using a CRISPR/Cas system. In some embodiments, a Cas nuclease and one or more guide RNAs are introduced into the cells to allow for genetic engineering. In some embodiments, the guide RNAs are specific for at least one of cytokine inducible SH2 containing protein (CISH), programmed death-1 (PD-1), and Fas receptor (FasR) and result in reduced activity of the protein. In some embodiments, the genetic engineering comprises contacting the cells with a vector comprising one or more exogenous nucleic acids operably linked to a promoter. In some embodiments, the one or more exogenous nucleic acids encode a Cas nuclease and/or one or more guide RNAs. In some embodiments, the one or more guide RNAs are targeted to one or more genes encoding a protein in the cells. In some embodiments, the one or more guide RNAs target one or more genes encoding a protein in the cells selected from: cytokine inducible SH2 containing protein (CISH), programmed death-1 (PD-1), and Fas receptor (FasR). In some embodiments, the cells are genetically engineered by contact with a vector encoding a transposon. In some embodiments, the transposon is a TcBuster™ transposon. In some embodiments, the population of polyclonal GDTCs are expanded at least 100-fold. In some embodiments, the population of polyclonal GDTCs are expanded at least 1000-fold. In some embodiments, the population of polyclonal GDTCs are expanded at least 3000-fold. In some embodiments, the anti-gammadelta T cell receptor antibody is linked to a solid support. In some embodiments, the solid support comprises the surface of the vessel in which the GDTCs are cultured in step b). In some embodiments, the solid support comprises beads. In some embodiments, the method further comprises contacting the cells with a FasL blocking reagent. In some embodiments, step (a) to activate the GDTCs is about 1-3 days. In some embodiments, step (c) to re-stimulate the GDTCs is about 1-3 days. In some embodiments, step (b) to expand the GDTCs is greater than about 10 days, optionally about 11 days.

In another aspect of the current disclosure, kits for expanding polyclonal gammadelta T cells (GDTCs) are provided. In some embodiments, the kits comprise: i) anti-gammadelta T cell receptor (GDTCR) antibody; ii) anti-CD28 antibody; and iii) one or more, two or more, or all three of IL 2, IL-7, IL-15 cytokines. In some embodiments, the one or more of IL-2, IL-7, and IL-15 cytokines are human IL-2, IL-7, or IL-15. In some embodiments, the anti-gammadelta T cell receptor antibody is linked to a solid support. In some embodiments, the solid support comprises beads. In some embodiments, the kits further comprise: iv) a FasL blocking reagent.

In another aspect of the current disclosure, methods of treating a cell proliferative disease or disorder in a subject in need thereof are provided. In some embodiments, the methods comprise: administering a GDTC made by methods comprising: a) incubating GDTCs in media comprising: i) anti-gammadelta T cell receptor (GDTCR) antibody; ii) anti-CD28 antibody; and iii) one or more of IL-2, IL-7, IL-15 to activate the GDTCs; b) culturing the GDTCs to expand the GDTCs; and c) re-stimulating the GDTCs of step (b) by incubating the GDTCs in medium comprising: i) anti-gammadelta T cell receptor antibody; and ii) anti-CD28 antibody to re-stimulate the polyclonal GDTCs; to a subject in need thereof to treat the cell proliferative disease or disorder. In some embodiments, the GDTCs are not cultured in the presence of a bisphosphonate or in the presence of feeder cells. In some embodiments, the methods further comprising at step (b) genetically engineering the cells to alter the expression of one or more proteins in the GDTCs. In some embodiments, the cells are genetically engineered using a CRISPR/Cas system. In some embodiments, a Cas nuclease and one or more guide RNAs are introduced into the cells to allow for genetic engineering. In some embodiments, the guide RNAs are specific for at least one of cytokine inducible SH2 containing protein (CISH), programmed death-1 (PD-1), and Fas receptor (FasR) and result in reduced activity of the protein. In some embodiments, the genetic engineering comprises contacting the cells with a vector comprising one or more exogenous nucleic acids operably linked to a promoter. In some embodiments, the one or more exogenous nucleic acids encode a Cas nuclease and/or one or more guide RNAs. In some embodiments, the one or more guide RNAs are targeted to one or more genes encoding a protein in the cells. In some embodiments, the one or more guide RNAs target one or more genes encoding a protein in the cells selected from: cytokine inducible SH2 containing protein (CISH), programmed death-1 (PD-1), and Fas receptor (FasR). In some embodiments, the cells are genetically engineered by contact with a vector encoding a transposon. In some embodiments, the transposon is a TcBuster™ transposon. In some embodiments, the population of polyclonal GDTCs are expanded at least 100-fold. In some embodiments, the population of polyclonal GDTCs are expanded at least 1000-fold. In some embodiments, the population of polyclonal GDTCs are expanded at least 3000-fold. In some embodiments, the anti-gammadelta T cell receptor antibody is linked to a solid support. In some embodiments, the solid support comprises the surface of the vessel in which the GDTCs are cultured in step b). In some embodiments, the solid support comprises beads. In some embodiments, the method further comprises contacting the cells with a FasL blocking reagent. In some embodiments, step (a) to activate the GDTCs is about 1-3 days. In some embodiments, step (c) to re-stimulate the GDTCs is about 1-3 days. In some embodiments, step (b) to expand the GDTCs is greater than about 10 days, optionally about 11 days. In some embodiments, the cell proliferative disease or disorder is selected from: bone cancer, brain cancer, breast cancer, cervical cancer, cancer of the larynx, lung cancer, pancreatic cancer, prostate cancer, skin cancer, cancer of the spine, stomach cancer, uterine cancer, hematopoietic cancer, and/or lymphoid cancer. In some embodiments, the cell proliferative disease or disorder comprises: acute myelogenous leukemia (AML), acute lymphoblastic leukemia (ALL), myelodysplastic syndromes (MDS), non-Hodgkin lymphoma (NHL), chronic myelogenous leukemia (CML), Hodgkin's disease, or multiple myeloma.

In another aspect of the current disclosure, populations of expanded genetically modified polyclonal gammadelta T cells (GDTCs), wherein the GDTCs comprise at least 10 distinct gammadelta T cell receptor (GDTCR) clones are provided. In some embodiments, the population comprises at least 50 GDTCR clones. In some embodiments, the GDTCs comprise a modification in a gene selected from: cytokine inducible SH2 containing protein (CISH), programmed death-1 (PD-1), and Fas receptor (FasR). In some embodiments, the population of GDTCs comprises less than about 5% of GDTCs expressing CD27 on their surface and greater than about 95% of GDTCs expressing CD45RO on their surface, as measured by flow cytometry. In some embodiments, the population comprises greater than about 1 million, greater than about 10 million, or greater than about 100 million GDTCs.

In another aspect of the current disclosure, further methods of treating a cell proliferative disease or disorder in a subject in need thereof are provided. In some embodiment, the methods comprise: administering a population of polyclonal gammadelta T cells (GDTCs), wherein the GDTCs comprise at least 10 distinct gammadelta T cell receptor (GDTCR) clones, to a subject in need thereof to treat the cell proliferative disease or disorder. In some embodiments, the population comprises at least 50 GDTCR clones. In some embodiments, the GDTCs comprise a modification in a gene selected from: cytokine inducible SH2 containing protein (CISH), programmed death-1 (PD-1), and Fas receptor (FasR). In some embodiments, the population of GDTCs comprises less than about 5% of GDTCs expressing CD27 on their surface and greater than about 95% of GDTCs expressing CD45RO on their surface, as measured by flow cytometry. In some embodiments, the population comprises greater than about 1 million, greater than about 10 million, or greater than about 100 million GDTCs. In some embodiments, the cell proliferative disease or disorder is selected from: bone cancer, brain cancer, breast cancer, cervical cancer, cancer of the larynx, lung cancer, pancreatic cancer, prostate cancer, skin cancer, cancer of the spine, stomach cancer, uterine cancer, hematopoietic cancer, and/or lymphoid cancer. In some embodiments, the cell proliferative disease or disorder comprises: acute myelogenous leukemia (AML), acute lymphoblastic leukemia (ALL), myelodysplastic syndromes (MDS), non-Hodgkin lymphoma (NHL), chronic myelogenous leukemia (CML), Hodgkin's disease, or multiple myeloma. In some embodiments, the cells comprise greater than about 1 million, greater than about 10 million, or greater than about 100 million GDTCs.

### BRIEF DESCRIPTION OF THE FIGURES

FIG. 1. Expansion of total and Vd2-positive GDTCs during in vitro culture of peripheral blood mononuclear cells (PBMC) with zoledronate (Zo) and IL-2.

FIG. 2. (A) Impact of stimulatory cytokines and zoledronate exposure on T cell expansion and transfection efficiency. PBMCs cultured under various stimulation conditions were electroporated on day 4, then evaluated seven days post-electroporation (EP). (B) Impact of stimulatory cytokines and zoledronate exposure on T cell expansion in Zo-stimmed PBMCs electroporated on day 7, evaluated seven days post-EP. (C) B2M knockout in Zo-stimmed PBMCs electroporated on day 7, with or without RNase inhibitor.

FIG. 3. (A) Impact of RNase inhibitor and zoledronate-containing recovery media on T cell expansion and transfection efficiency in Zo-stimmed, day 4-electroporated PBMCs one day post-EP. (B) Impact of RNase inhibitor and zoledronate-containing recovery media on B2M knockout in Zo-stimmed, day 4-electroporated PBMCs seven days post-EP. (C and D) Impact of RNase inhibitor and zoledronate-containing recovery media on T cell expansion and transfection efficiency in Zo-stimmed, day 7-electroporated PBMCs, evaluated at one and seven days post-EP. (E and F) Viability, cell size, and total cells evaluated at day 11 for zo-stimmed PBMCs electroporated on days 4 (+/−Zo in recovery media) and 7 (+/− day 4 re-stim, +/−Zo in recovery media).

FIG. 4. (A) Impact of electroporation voltage on total cells, viability, and cell size in zo-stimmed PBMCs electroporated on day 4, evaluated one day post-electroporation. (B) Impact of electroporation voltage on T cell expansion and transfection efficiency in zo-stimmed PBMCs electroporated on day 4, evaluated one day post-electroporation.

FIG. 5. Impact of plate-bound stimulatory antibodies, with and without soluble CD28, on gamma delta T cell expansion and lymphocyte distribution in PBMCs. Evaluated on day 7 post-stim.

FIG. 6. (A and B) Impact of day 4 and day 11 re-stims on expansion of purified GDTCs stimulated with plate-bound Pan-GDTCR+soluble CD28, plate-bound OKT3+soluble CD28, and dynabeads. (C) Impact of GREX culture on total cell counts at day 24. Day-11 re-stimmed cells were placed in 24-well GREX wells at 8×10⁵ cells/well on day 13. (D) total number of cells at D24 (E) Vd1 expression at D24 in GREX-cultured cells.

FIG. 7. (A and B) Phenotyping and count data for purified GDTCs stimmed with plate-bound Pan-GDTCR+soluble CD28 and electroporated on day 3. Electroporations were performed at 3×10⁵ cells/condition.

FIG. 8. (A) Total cells on day 11 for purified Pan-GDTCR+soluble CD28-stimmed GDTCs electroporated with a CD19-GFP CAR and Snoke on days 2, 4, or 6. Electroporations were performed at 3×10⁵ cells/condition. The adjustment accounts for growth in the day 4 and 6 samples that occurred between day two and their respective electroporations. (B) Transfection efficiency on day 11 as a function of electroportion day.

FIG. 9. CISH knockout efficiency at day 11 for purified Pan-GDTCR+soluble CD28-stimmed GDTCs electroporated with ABE8e+CISH gRNA on day 2, either alone or in conjunction with a CD19-GFP CAR and Snoke. Selected populations were derived via exposure of unselected CAR+ populations to methotrexate.

FIG. 10. Cytotoxicity of D22 expanded GDTCs. (A) Cytotoxicity of D22 expanded GDTC after 24 hr co-culture with Raji target cells as measured by luciferase assay at indicated effector to target ratios. (B) Cytotoxicity of D22 expanded GDTC after 48 hr co-culture with Raji target cells as measured by luciferase assay at indicated effector to target ratios.

FIG. 11. Representative fold expansion of electroporated γδ T cells. Human γδ T cells were isolated from two healthy donors, then stimulated with plate-bound pan-GDTCR Ab and soluble CD28. After 48 hours, cells were given a “pulse” electroporation, in which no additional reagents were delivered. Cells were then cultured out to day 22, with a re-stimulation on day 11.

FIG. 12. Generalized methodology for Ab-based γδ T cell engineering and expansion. Human γδ T cells are isolated by negative selection from healthy donor PBMCs, then exposed to soluble CD28 and plate-bound pan-GDTCR Ab. After 36-48 hours, γδ T cells are electroporated with genome engineering reagents-including viral vectors, base editors, and transposons-then activated/expanded until day 11. Following a re-stimulation with soluble CD28 and pan-GDTCR Ab, cells are expanded until D22. Polyclonal γδ T cells are then harvested, frozen, and prepared for downstream applications. Typical reagent volumes include 2 ng/mL soluble CD28 and 1.25 ng/cm² plate-bound pan-GDTCR Ab.

FIG. 13. Comparing zoledronate and Ab-based γδ T cell stimulation. Peripheral blood γδ T cells (N=2 human donors) were either cultured with zoledronate or isolated and stimulated with pan-GDTCR Ab+soluble CD28. Cells were then electroporated with Cas9 mRNA and PD1 sgRNA on day 2. (A) PD1 gene knockout on day 11, quantified by indel formation. (B) Fold expansion on day 11. Pulse populations were electroporated without Cas9 or sgRNA. (C) TCR heterogeneity in CD3+ cells on day 11. Vd1/Vd2 expression was evaluated by flow cytometry.

FIG. 14. Fold expansion of Ab-stimulated γδ T cells at day 11 and day 22. Pulse γδ T cells were exposed to pan-GDTCR Ab+soluble CD28, then either harvested or re-stimulated at day 11. Re-stimulated cells were then expanded until day 22. N=2 human donors.

FIG. 15. Use of the TcBuster™ transposon (TcB) to facilitate CAR integration. TcB is a DNA transposon that allows for stable nonviral integration of large (>10 kb) constructs. For γδ T cell engineering, TcB is delivered as mRNA, along with a 5.4 kb nanoplasmid (NP) transposon vector containing a CD19 CAR. Following electroporation, TcB protein binds to conserved flanking sites on the NP, excises the CAR cassette, then mediates its ligation into the genome to achieve stable CAR integration and expression.

FIG. 16. CAR Construct Design. Anti-CD19 CAR expression was driven by the MND synthetic promoter. The CAR sequence was followed by a mutant dihydrofolate reductase (DHFR) gene, which acts as a selectable marker by conferring resistance to methotrexate. EGFP was included as a fluorescence reporter. 5′ and 3′ TcB inverted terminal repeats (ITR) were included to promote construct integration. All proteins were separated by 2A sequences.

FIG. 17. Methotrexate exposure enriches populations of CAR+γδ T cells. γδ T cells were electroporated with CD19 CAR nanoplasmid and TcBuster transposon mRNA on day 2, then selected at different timepoints with methotrexate. In addition to a GFP reporter, a mutant DHFR gene was included in the CAR construct to confer resistance to methotrexate. (A) CAR expression of γδ T cells (N=2 human donors) at day 11. Cells in the “SEL” population were exposed to methotrexate on day 6. “Pulse” cells were electroporated but received no editing reagents. (B) CAR expression of γδ T cells at day 22. S/U notation indicates the presence(S) or absence (U) of selection on days 6 and 17. GFP expression was evaluated by flow cytometry. (C) Total fold expansion of differentially selected cells at day 22.

FIG. 18. Ab-stimulated CAR γδ T cells exhibit potent cytotoxicity against cancer cells. Pan-GDTCR-stimulated γδ T cells were electroporated with a CD19 CAR on day 2, then selected at different timepoints with methotrexate. γδ T cells were harvested at day 22, and exposed to Raji-Luc cancer cells at 3:1. 1:1, and 1:3 effector; target (ET) ratios. Cytotoxicity was quantified by the reduction of luciferin luminosity at 48 hours. N=2 human donors, with 3 technical replicates per donor.

FIG. 19. Targeting CISH, PD1, and FasR to enhance anti-tumor activity. γδ T cells express regulatory proteins that dampen their anti-tumor activity. CISH is a negative regulator of IL-15 signaling and reduces γδ T cell proliferation following activation. PD1 is an immune checkpoint protein whose cognate ligand, PDL1, is expressed by many tumor cells. Ligation of PD1 by PDL1 leads to downstream inhibition of the γδ T cell. γδ T cells also co-express high levels of FasL and FasR, reducing activation-induced expansion due to self-apoptotic activity and diminishing functionality in solid tumor settings.

FIG. 20. Highly efficient base editor knockout of target genes in γδ T cells. γδ T cells (N=2 human donors) were isolated and stimulated using pan-GDTCR+soluble CD28. After 48 hours, cells were electroporated with TcB, CD19 CAR NP, and ABE8e mRNA, along with sgRNAs for CISH, PD1, and FasR. The targeting sequences for CISH, PD1 and FasR are SEQ ID NOs: 7, 8, and 9, respectively. CAR+γδ T cells were selected on day 6. Genomic DNA was sequenced on day 11 to evaluate editing. N=2 human donors.

FIG. 21. Base editor-engineered CAR γδ T cells display potent anti-cancer activity. Pan-GDTCR-stimulated γδ T cells were engineered with a CD19 CAR and different gene knockout combinations on day 2, then selected on day 6 with methotrexate. γδ T cells were harvested at day 22 and exposed to Raji-Luc cancer cells at 1:1. 1:3, and 1:9 E:T ratios. At intervals of 48 hours, γδ T cells from the 1:1 conditions were re-plated with fresh Raji-Luc cells at 1:1. 1:3, and 1:9 E:T ratios. (A) Cytotoxicity after I round of Raji-Luc exposure. “TX-100” cells were exposed to 1% Triton X-100 as a positive control for cell lysis. (B) Cytotoxicity after 3 rounds of Raji-Luc exposure. (C) Cytotoxicity after 5 rounds of Raji-Luc exposure. (D) During rounds 1 and 2, CISH, Fas, and PD1 triple knockout produced a statistically significant increase in cytotoxicity. Cytotoxicity was quantified by the reduction of luciferin luminosity at 48 hours. N=2 human donors, with 3 technical replicates per donor.

FIG. 22. Vδ1 and Vδ2 TCR expression in Ab-stimulated cord blood and peripheral blood populations. γδ T cells were isolated from one peripheral blood (LP4) and one umbilical cord blood donor (UB4), then expanded with pan-GDTCR Ab+soluble CD28. On day 22, relative Vδ1 and Vδ2 TCR expression was evaluated for each population by flow cytometry. Vδ1/Vδ2 expression is displayed within a CD3+ gate.

FIG. 23. Freshly isolated cord blood and peripheral blood γδ T cells exhibit differences in maturity. γδ T cells were isolated from one peripheral blood (LP4) and one cord blood donor (UB4) via negative selection, then characterized using flow cytometry. CD27 is a costimulatory receptor expressed predominantly on naïve T cells, while CD45RA is a surface marker expressed on naïve T cells. CD27/CD45RA expression is displayed within a CD3+ gate.

FIG. 24. Ab stimulation promotes maturation of cord blood γδ T cells. γδ T cells were isolated from one peripheral blood (LP4) and one cord blood donor (UB4) via negative selection, then expanded with pan-GDTCR Ab+soluble CD28. On day 22, CD27, CD45RA, and CD45RO expression was quantified by flow cytometry. CD45RA is expressed on naïve T cells, while its splice isoform, CD45RO, is expressed on mature T cells. CD27 is expressed predominantly on naïve T cells. (A) CD27/CD45RO expression in peripheral and cord blood γδ T cells possessing Vδ1 or Vδ2 TCRs. (B) CD45RA/CD45RO expression in peripheral and cord blood γδ T cells possessing Vol or Vδ2 TCRs.

FIG. 25. Ab-stimulated γδ T cells possess activated phenotypes. γδ T cells were isolated from one peripheral blood (LP4) and one cord blood donor (UB4) via negative selection, then expanded with pan-GDTCR Ab+soluble CD28. On day 22, CD56 expression was quantified by flow cytometry. CD56 is a surface marker expressed on activated natural killer cells and γδ T cells. CD56 expression is displayed within either Vδ1+ or Vδ2+ gates.

FIG. 26. Expansion of Ab-stimulated cord blood and peripheral blood γδ T cells. γδ T cells were isolated from one peripheral blood (LP4) and one cord blood donor (UB4) via negative selection, then expanded with pan-GDTCR Ab+soluble CD28. A re-stimulation was performed on day 11.

### DETAILED DESCRIPTION

The present invention describes a defined, feeder-free methodology for potent and selective expansion of a polyclonal gamma delta T cell (GDTC or γδ T cells) population from peripheral blood, expanded populations of these cells and methods of using these expanded cells. The novel two-stage expansion protocol described herein is compatible with cGMP manufacture, achieves remarkably high levels of expansion (greater than 3000-fold), and is highly compatible with non-viral genetic engineering approaches such as CRISPR/Cas9 and transposon mediated engineering to allow for production of large quantities of engineered cells for therapeutic use. In some embodiments, the methods described are capable of producing large amounts of polyclonal GDTC (including engineered GDTC) for use in therapeutic methods, described more herein.

In one aspect of the current disclosure, methods of expanding a population of polyclonal gammadelta T cells (GDTCs) in vitro are provided. The methods include incubating GDTCs in media comprising anti-gammadelta T cell receptor (GDTCR) antibody, an anti-CD28 antibody and one or more of IL-2, IL-7, and IL-15 to activate the GDTCs. The initial incubation step may be carried out for 1-3 days or more. Thus the initial activation/incubation step may be 1, 2, 3, 4, 5, 6, 7, 8, 9 or even 10 days, but 1-3 days was used in the examples. After incubation, the GDTCs are cultured to allow for expansion of the GDTCs. The expansion phase is at least 3 days and may be up to two weeks or any amount of time between 3 and 14 days, but 10-11 days was used in the examples. The resulting expanded GDTCs are then re-stimulated by incubating the GDTCs in medium comprising an anti-gammadelta T cell receptor antibody and an anti-CD28 antibody. The re-stimulation is generally for about 1-3 days to re-stimulate the polyclonal GDTCs in culture. The re-stimulation may be for 1, 2, 3, 4, 5, 6, 7, 8, 9, 10, 11 or even more days. The method provides a superior method than prior methods as no feeder cells are needed and the cells are cultured without addition of bisphosphonate. The cells can be expanded in vitro at least 100-fold, alternatively at least 1000-fold, or at least 3000-fold using the methods provided while maintaining their polyclonal TCR repertoire.

Gammadelta T cells (γδ T cells, GDTCs) are T cells that express a unique T-cell receptor (TCR) composed of one γ-chain and one δ-chain. Gammadelta T cells are of low abundance in the body and are involved in the initiation and propagation of immune responses. GDTCs are capable of infiltrating solid tumors and directly killing transformed cells in a largely MHC-independent fashion via recognition of stress-induced antigens and metabolites. Since GDTCs are the fraction of tumor infiltrating lymphocytes most highly correlated with positive outcomes from anti-cancer immunotherapies, GDTCs may be better than αβ T cells (alpha-beta T cells) for infiltrating solid tumor microenvironments and efficient tumor-cell killing. GDTCs have innate and adaptive qualities exhibiting a range of effector functions, including cytolysis upon cell contact. T cells expressing TCRα/TCRβ heterodimers compose approximately 95% of peripheral blood (PB) T cells and recognize peptides in the context of major histocompatibility complex (MHC). In contrast, GDTCs are infrequent, only comprising about 1-5% of T cells in peripheral blood. The TCR γδ ligands further are recognized independent of MHC, thus limiting the risk of graft-versus host disease in an allogeneic setting. Cancer cells express many conserved ligands for γδ T cell receptor, and therefore the ability to propagate γδ T cells from small starting numbers while maintaining a polyclonal repertoire of γδ TCRs has appeal for therapeutic application. Given the low frequencies of γδ T cells in blood, the present invention provides a method compliant with good manufacturing practice (cGMP) to expand polyclonal γδ T cells. In some embodiments, these γδ T cells provide an ideal platform for the development of immunotherapies against blood and solid tumors.

In some embodiments, the GDTCs generated by the methods disclosed herein are used as allogeneic therapies for cancer, infectious disease, and for gene therapy, e.g., for delivery of therapeutic proteins. Suitable methods of using the cells for therapy are known and understood in the art. In one embodiment, the methods described herein are able to produce large quantities of γδ T cells from allogeneic (e.g., unrelated and healthy donors) that can be administered as an off-the shelf therapy. In some embodiments, the therapy may be for hematologic or solid tumors.

Preferably, the GDTC made by the methods described herein are polyclonal. The term “polyclonal” refers to a population of cells that are derived from many clones of GDTCs expressing different somatically rearranged gamma delta T cell receptors (GDTCRs). In some embodiments, a clone, i.e., a clone of GDTCs, is defined by the expression of a particular gamma delta T cell receptor (GDTCR). Put another way, all GDTCs that belong to a particular clone will have the same GDTCR sequence. Previous methods of expanding GDTCs used aminobisphosphonates, e.g., zoledronate. However, such methods are only capable of expanding Vg9Vd2 subsets and, further, require culture with mononuclear cells (MNCs) as feeder cells for efficient activation. By contrast, the methods of the current disclosure efficiently expand polyclonal GDTCs, i.e., more than Vg9Vd2 subset of GDTCs. In some embodiments, a polyclonal population of cells comprises 5 or more clones. In some embodiments, a polyclonal population of cells comprises 10 or more clones.

In preferred embodiments, the methods described herein to expand GDTCs in in vitro culture do not use a feeder cell line. As used herein, “feeder cell” or “feeder cell line” refers to cells or a particular line of cells that support the growth and/or differentiation of another type of cell. Accordingly, feeder cells may be irradiated or otherwise treated to prevent the feeder cell line from dividing. In the current disclosure, the methods do not require feeder cells or a feeder cell line to support the growth and activation or differentiation of GDTCs. Therefore, the methods of the current disclosure represent an improvement over existing methods.

The term incubating and culturing and variations thereof are used herein interchangeably to refer to the culturing of the cells in suitable medium (i.e., cell culture). Both incubating and culturing can further include additional factors that provide for a biological change to take place. In some embodiments, GDTCs are incubated in a medium comprising cytokines and antibodies that activate signaling pathways such as, for example, T cell receptor (TCR) signaling, or CD28 (co-stimulatory) signaling. In some embodiments, “cell culture” refers to the culturing of mammalian cells, wherein the cells are cultured in an artificial medium that supports their growth and, in some cases, differentiation. Methods of culturing cells are widely known in the art. Cells of the present disclosure are cultured in a medium that stimulates “expansion” of the cells. As used herein, “expansion” refers to an increase in cell number that may also coincide with differentiation of the cells. In some embodiments, the GDTCs are stimulated and expanded for about 11 days, whereupon the GDTCs are re-stimulated and allowed to expand further for, in one embodiment, about 11 days, about 12 days, about 13 days, about 14 days, about 15 days. Thus, in some embodiments, the entire method requires at least 22 days with expansion taking place 11 days after the initial activation and another 11-day period of re-stimulation taking place after the expansion period. However, it will be apparent to one of skill in the art that modifications to the expansion times may be made without significantly affecting the method of polyclonal GDTCs expansion. For example, the inventors contemplate that the GDTCs may be further restimulated and cultured.

The GDTCs for use in the methods may be isolated from peripheral blood mononuclear cells (PBMCs) or umbilical cord blood, otherwise known as “cord blood,” that have been removed from a subject or a donor. Methods of obtaining unexpanded/uncultured PBMCs from blood or leukocytes from cord blood, each of which comprise gammadelta T cells, are routine and known in the art. In some embodiments, the donor is a healthy donor. In other embodiments, the subject is a subject with a condition and who will be treated with the GDTCs after expansion.

GDTCs can be isolated according to any appropriate method. For instance, wild-type GDTCs can be isolated from peripheral blood mononuclear cells (PBMCs) or cord blood. PBMCs can be obtained from peripheral blood or cord blood using any appropriate technique such as, for example, an ACK-lysis buffer protocol. For example, GDTCs can be isolated using a commercially available kit such as the Easy Sep Human Gamma/Delta T Cell Isolation Kit 20) from StemCell Technologies. In other cases, GDTCs can be isolated by plating PBMCs in a culture medium containing Concanavalin A, IL-2, and IL-4 for about 1 week. Cells are further cultured in a culture medium that does not contain Concanavalin A for an additional 7 days. Another isolation method comprises plating PBMCs in a culture medium containing Zolendronic Acid and IL-2 for about 2 days. The cells can be further cultured in a medium that 25 does not contain Zolendronic Acid for an additional 12 days. In some cases, percent purity of the isolated γδ T cell population is determined using flow cytometry. Magnetic cell sorting, or another cell sorting method. In some embodiments, GDTCs are isolated using a negative selection method, i.e., wherein the method does not modify the cells by, for example, attaching antibodies to the surface of the cell. For example, one method of negative selection includes, 30) but is not limited to, depleting NK cells from the culture issuing CD56 antibodies or CD56 beads (e.g., magnetic beads). In some embodiments, GDTCs are isolated using a positive selection method, i.e., wherein the method modifies the cells by attaching, for example, antibodies to the surface of the cell. In another embodiment, the cells are isolated using magnetic beads or flow cytometry cell sorting (fluorescence activated cell sorting (FACS)). Suitable methods of isolating cells with specific cell surface markers are known in the art. In one example, the GDTCs are isolated using antibodies specific for TCRγδ, alone or in combination with anti-CD3 monoclonal antibodies (mAbs). In some embodiments, GDTCs are isolated using a method comprising: depletion of αβ T cells, followed by sorting for CD3+ cells. In some embodiments, the foregoing isolation strategy results in greater than 90%, greater than 91%, greater than 92%, greater than 93%, greater than 94%, greater than 95%, greater than 96%, greater than 97%, greater than 98%, or greater than 99% purity of GDTCs. In some embodiments, the reagents used to isolate GDTCs are good manufacturing procedure (GMP) compatible, e.g., the reagents do not comprise feeder cells or comprise bacterial endotoxin, e.g., lipopolysaccharides.

In the methods of the current disclosure, gamma delta T cells (GDTCs) are incubated with one or more of IL-2, IL-7, and IL-15. In some embodiments, GDTCs are incubated with two or more of IL-2, Il-7, and IL-15. In some embodiments, GDTCs are incubated with all three of IL-2, IL-7, and IL-15. In one example, GDTCs are incubated with IL-2. In another example, GDTCs are incubated with IL-7. In another example, GDTCs are incubated with IL-15. In some embodiments, GDTCs are incubated with IL-2 and IL-7. In some embodiments, GDTCs are incubated with IL-2 and IL-15. In some embodiments, GDTCs are incubated with IL-7 and IL-15. In some embodiments, GDTCs are incubated with IL-2, IL-7, and IL-15.

As used herein, “IL-2” refers to the cytokine interleukin 2 which is produced by T cells in response to a variety of signals. IL-2 has pleiotropic effects on the immune system. The amino acid sequence for human IL-2 is as follows (SEQ ID NO: 1). The amino acid sequence for mouse IL-2 is as follows (SEQ ID NO: 2). In some embodiments, the concentration of IL-2 is about 100 units/ml to about 10000 units/ml. In some embodiments, the concentration of IL-2 is about 300 units/ml to about 1000 IU/ml, e.g., about 300 units/ml to about 400 units/ml, about 300 units/ml to about 500 units/ml, about 300 units/ml to about 600 units/ml, about 300 units/ml to about 700 units/ml, about 300 units/ml to about 800 units/ml, about 300 units/ml to about 900 units/ml, about 400 units/ml to about 500 units/ml, about 400 units/ml to about 600 units/ml, about 400 units/ml to about 700 units/ml, about 400 units/ml to about 800 units/ml, about 400 units/ml to about 900 units/ml, about 400 units/ml to about 100 units/ml, about 500 units/ml to about 600 units/ml, about 500 units/ml to about 700 units/ml, about 500 units/ml to about 800 units/ml, about 500 units/ml to about 900 units/ml, about 500 units/ml to about 1000 units/ml, about 600 units/ml to about 700 units/ml, about 600 units/ml to about 800 units/ml, about 600 units/ml to about 900 units/ml, about 600 units/ml to about 1000 units/ml, about 700 units/ml to about 800 units/ml, about 700 units/ml to about 900 units/ml, about 700 units/ml to about 1000 units/ml, about 800 units/ml to about 900 units/ml, about 800 units/ml to about 1000 units/ml, about 900 units/ml to about 1000 units/ml. In some embodiments, the concentration of IL-2 is about 100 units/ml, about 200 units/ml, about 300 units/ml, about 400 units/ml, about 500 units/ml, about 600 units/ml, about 700 units/ml, about 800 units/ml, about 900 units/ml, or about 1000 units/ml.

As used herein, “IL-7” refers to the cytokine interleukin 7. Human IL-7 has the following amino acid sequence (SEQ ID NO: 3). Mouse IL-7 has the following amino acid sequence (SEQ ID NO: 4). In some embodiments, the concentration of IL-7 is about 0.5 ng/ml to about 50 ng/ml. In some embodiments, the concentration of IL-7 is about 0.5 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml about 10 ng/ml. In some embodiments, the concentration of IL-7 is about 5 ng/ml.

As used herein, “IL-15” refers to the cytokine interleukin 15. Human IL-15 has the following amino acid sequence (SEQ ID NO: 5). Mouse IL-15 has the following amino acid sequence (SEQ ID NO: 6). In some embodiments, the concentration of IL-15 is about 0.5 ng/ml to about 50 ng/ml. In some embodiments, the concentration of IL-15 is about 5 ng/ml. In some embodiments, the concentration of IL-15 is about 0.5 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml about 10 ng/ml.

As used herein, “anti-T cell receptor gamma delta antibody” refers to an antibody that is specific for gamma delta T cell receptor. Anti-T cell receptor gamma delta antibodies stimulate GDTCs by productively ligating the GDTC receptor (gdTCR), i.e., the antibodies are agonistic for the GDTCR. Suitable anti-gdTCR antibodies are known in the art, for example, TCRγ/δ Antibody, anti-human, REAfinity™ available from Miltenyi Biotec.

In some embodiments, the anti-T cell receptor gamma delta antibody is linked to a solid support. Suitable solid supports include, but are not limited to, beads (e.g., a colloidal particle, a metallic nanomaterial, a nanoparticle, a nanoplate, a nanoshell, a nanorod, a latex bead, polystyrene, polycarbonate, polyacrylate, PVDF, or PMMA, etc.), glass or plastic tissue culture surface, culture vessel or culture plate surfaces. Suitable support materials further include, but are not limited to, those supports that are typically used for solid phase chemical synthesis, e.g., polymeric materials (e.g., polystyrene, polyvinyl acetate, polyvinyl chloride, polyvinyl pyrrolidone, polyacrylonitrile, polyacrylamide, polymethylmethacrylate, polytetrafluoroethylene, polyethylene, polypropylene, polyvinylidene fluoride, polycarbonate, divinylbenzene styrene-based polymers), agarose (e.g., Sepharose®), dextran (e.g., Sephadex R), cellulosic polymers and other polysaccharides, silica and silica-based materials, glass, and functionalized glasses, ceramics, and such substrates treated with surface coatings, e.g., with microporous polymers (particularly cellulosic polymers such as nitrocellulose), microporous metallic compounds (particularly microporous aluminum), antibody-binding proteins (available from Pierce Chemical Co., Rockford Ill.), bisphenol A polycarbonate, or the like. The substrate chosen may be coated with or complexed with an antibody binding protein such as protein A or protein G or another agent capable of attaching the antibody to the substrate. Such agents are known and available to those skilled in the art.

As used herein, “anti-CD28 antibody” refers to an antibody that is specific for the CD28 receptor and causes productive ligation with the CD28 receptor which is believed to act as co-stimulation for activation of GDTCs. Suitable anti-CD28 antibodies are known in the art, for example, CD28 Monoclonal Antibody (CD28.2), Functional Grade available from eBioscience™.

As used herein, “stimulating”, or grammatical variations thereof, refers to activating receptors or signaling pathways that results in a biological change in cells. In some embodiments, GDTCs are stimulated to be activated. In some embodiments, GDTCs are “re-stimulated” after an initial stimulation. As used herein, “re-stimulation” refers to stimulating cells subsequently to an initial stimulation. In some embodiments, re-stimulation may be performed in the same manner as the initial stimulation. In some embodiments, re-stimulation is performed using different conditions than the initial stimulation of the cells. In some embodiments, cells are stimulated or re-stimulated with anti-gamma delta T cell receptor (GDTCR) antibodies. In some embodiments, GDTCs are further stimulated, in addition to stimulation or re-stimulation with anti-GDTCR antibodies, with anti-CD28 antibodies, IL-2, IL-7, and IL-15, or any combination of anti-CD28 antibodies, IL-2, IL-7, and IL-15.

The GDTCs may be engineered or genetically engineered to alter the expression of one or more proteins within the GDTCs. Altering the expression of a protein includes expressing a protein not normally expressed in GDTCs, knocking out a protein normally expressed in GDTCs or decreasing or increasing the expression of a protein in GDTCs. In some embodiments, the GDTCs are genetically engineered to alter the expression of one or more transcripts. A γδ T cell that contains an exogenous, recombinant, synthetic, and/or otherwise modified polynucleotide is considered to be an engineered or “genome edited” cell. Genetically editing or modifying a cell refers to modifying cellular nucleic acid within a cell, including genetic modifications to endogenous and/or exogenous nucleic acids within the cell. Genetic modifications can comprise deletions, insertions, integrations of exogenous DNA, gene correction and/or gene mutation.

In one embodiment, the method comprises contacting the cells with a vector comprising one or more exogenous nucleic acids operably linked to a promoter. In some embodiments, the one or more exogenous nucleic acids encode a Cas nuclease and/or one or more guide RNAs. Alternatively, the Cas nuclease may be introduced to the cells as a protein and the one or more guide RNAs may be introduced to the cells directly or via a DNA encoding the guide RNA. In some embodiments, the cell has one or more genes knocked-out. Methods of genetically modifying the cells are described in more detail below; but methods of genetic engineering are available to those of skill in the art.

Suitable vectors for use with the present invention comprise a promoter operably connected to a polynucleotide sequence encoding the one or more peptides or transcripts to be expressed within the cell. The vectors may also comprise appropriate control sequences that allow for translational regulation in a GDTC. In some embodiments, the vectors further comprise nucleic acid sequences encoding one or more agents or tags. In some embodiments, the vectors further comprise additional regulatory sequences, such as signal sequences. As used herein, the term “vector” refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors” (or simply, “vectors”). The term vector encompasses “plasmids”, the most commonly used form of vector. Plasmids are circular double-stranded DNA loops into which additional DNA segments (e.g., those encoding one or more peptides) may be ligated. However, other forms of expression vectors, such as viral vectors (e.g., replication defective retroviruses, adenoviruses and adeno-associated viruses), may also be used with the present invention. In some embodiments, the vectors of the present invention further comprise heterologous backbone sequence. As used herein, “heterologous nucleic acid sequence” refers to any nucleic acid sequence, for example, a bacterial, viral, or other nucleic acid sequence that is not naturally found in a the cell. Heterologous backbone sequences may be necessary for propagation of the vector and/or expression of the encoded peptide. Many commonly used expression vectors and plasmids contain non-human nucleic acid sequences, including, for example, CMV promoters.

The methods described herein are capable of producing an expanded population of polyclonal GDTCs. In some embodiments, the GDTCs are expanded at least 100-fold. In other embodiments, the population of polyclonal GDTCs are expanded at least 1000-fold. In further embodiments, the population of polyclonal GDTCs are expanded at least 3000-fold.

In another embodiment, the present disclosure provides an expanded polyclonal GDTCs made by the method described herein. The GDTCs may be allogenic to a subject in which the cells may be used. Preferably, the methods described herein can be used to make an off-the shelf cellular population that can be used for therapeutic applications.

In another embodiment of the current disclosure, methods of expanding and genetically engineering a population of polyclonal gamma delta T cells (GDTCs) in vitro are provided. The methods include incubating GDTCs in media, which includes an anti-T cell receptor gamma delta antibody, an anti-CD28 antibody, and one or more of IL 2, IL-7, IL-15 to activate the GDTCs. After activation, the GDTCs are expanded in culture and during this culturing step the GDTCs are genetically engineered. In one embodiment, the cells are genetically engineered by contacting the cells with a vector comprising one or more nucleic acids operably linked to a promoter. In another embodiment, the GDTCs are engineered by introduction of a CRISPR/Cas nuclease and at least one guide RNA. After the expansion and genetic engineering of the cells, the cells are re-stimulated by incubating the GDTC in medium comprising anti-T cell receptor gamma delta antibody and anti-CD28 antibody.

As used herein, “engineered” or “genetic engineering” or “genome-edited” and grammatical variations thereof, refers to the deliberate modification of the characteristics of a cell by manipulating its genetic material. In some embodiments, the genome-edited γδ T cell includes a modification in a coding region of the genome (for example, a gene) or a noncoding region of the genome. In some embodiments, a portion of genomic information and/or a gene may be deleted. In some embodiments, a portion of genomic information and/or a gene may be added. In some embodiments, the genomic information and/or the gene that is added is exogenous. In some embodiments, “exogenous” genomic information or an “exogenous” gene may be genomic information or a gene from a non-gamma delta T cell. In some embodiments, “exogenous” genomic information or an “exogenous” gene may be artificially generated including, for example, nucleic acids encoding a chimeric antigen receptor (CAR) or a marker gene. In some embodiments, a portion of genomic information and/or a gene may be altered, for example, by a mutation. A mutation may include, for example, a point mutation, a frameshift mutation, etc. The GDTCs expanded in vitro described herein may be engineered or genetically altered to have a gene knocked out or knocked in. As used herein, “knocking out” or “knocked out” refers to polynucleotides whose sequences have been mutated such that a cell is substantially unable to produce either functional transcripts or peptides therefrom. In some embodiments, the polynucleotide that is knocked out is a gene encoded by the genomic DNA of the cell. “Genomic DNA” refers to the DNA of a genome of an organism including, but not limited to, the DNA of the genome of a bacterium, fungus, archean, plant, or animal. Several methods of knocking out genes and mutating polynucleotides are known in the art. In some embodiments, the “CRISPR Cas” or other gene editing system is used to induce mutations in the polynucleotide to knock out a gene. As used herein, “knocking in” or “knocked in” refers to polynucleotides whose exogenous sequence has been added to a cell and is capable of expressing a functional transcript or peptide therefrom. In some embodiments, the CRISPR Cas or other gene editing system is used to knock in a transcript or protein.

For example, the genome-edited γδ T cell may comprise a mutation in one or more genes encoding an inhibitory receptor, whereby expression of the inhibitory receptor is decreased, partially or fully. The one or more genes encoding an inhibitory receptor can be selected from IL-17A (Interleukin 17A), DGKA (Diacylglycerol Kinase Alpha), DGKZ (Diacylglycerol Kinase Zeta), PD1 (programmed cell death 1), TRGC1 (T-cell Receptor Gamma Constant-1), TRGC2 (T-cell Receptor Gamma Constant-2), TRDC (T-cell Receptor Delta Constant), PD-L1 (Programmed death-ligand 1; also known as CD274), and CISH (Cytokine-inducible SH2-containing protein), or any combination thereof. Other inhibitory receptor genes include, without limitation, CD94-NKG2A, NKG2A, TIGIT, a member of the KIR2DL family (for example, KIR2DL1; KIR2DL2; KIR2DL3; KIR2DL4; or KIRDL5), a member of the KIR3DL family (KIR3DL1; KIR3DL2; or KIR3DL3), KLRG1, LILR, 2B4 (CD48), CD96 (Tactile), LAIR1, KLB1 (CD161), CEACAM-1, SIGLEC3, SIGLEC7, SIGLEC9, HPK1, FAS, TGFbR2, and/or CTLA4.

In some cases, the genetically modified γδ T cell is further modified to express a chimeric antigen receptor. As used herein, the term “chimeric antigen receptor (CAR)” (also known in the art as chimeric receptors and chimeric immune receptors) refers to an artificially constructed hybrid protein or polypeptide comprising an extracellular antigen binding domain of an antibody (e.g., single chain variable fragment (scFv)) operably linked to a transmembrane domain and at least one intracellular domain. Generally, the antigen binding domain of a CAR has specificity for a particular antigen expressed on the surface of a target cell of interest. For example, a T cell can be engineered to express a CAR specific for a molecule expressed on the surface of a particular cell (e.g., a tumor cell, B-cell lymphoma). The antigen recognition region of the extracellular domain permits binding of the CAR to a particular antigen of interest, for example, an antigen present on a cell surface, and thereby imparts specificity to a cell expressing a CAR.

A DNA sequence that “encodes” a particular RNA is a DNA nucleic acid sequence that is transcribed into RNA. A DNA polynucleotide may encode an RNA (mRNA) that is translated into protein, or a DNA polynucleotide may encode an RNA that is not translated into protein (e.g., tRNA, rRNA, or a guide RNA; also called “non-coding” RNA or “ncRNA”). A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence (e.g., site-directed modifying polypeptide, or Cpf1 polypeptide) and/or regulate translation of an encoded polypeptide.

“Heterologous,” as used herein, means a nucleotide or polypeptide in a cell that is not its native cell. In some embodiments, heterologous nucleic acids, i.e., nucleic acids transferred from one source to a cell, comprise nucleic acids encoding reporters, for example, green fluorescent protein (GFP) or other similar fluorescent proteins known in the art. In some embodiments, heterologous nucleic acids comprise nucleic acids that encode a Cas nuclease. In some embodiments, the Cas nuclease is Cas9. In some embodiments, heterologous nucleic acids further comprise guide RNAs (gRNAs).

Exemplary methods of genetic modification (also referred to as “transformation”, or “transfection”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv Drug Deliv Rev. September 2012 13. pii: S0169-409X (12) 00283-9. doi: 10.1016/j.addr.2012.09.023), gene editing, and the like.

As used herein, “guide RNA” or “gRNA” refers to a specific RNA sequence that recognizes the target DNA region of interest and directs Cas nuclease there for editing. The gRNA is made up of two parts: crispr RNA (crRNA), a 17-20 nucleotide sequence complementary to the target DNA, and a tracr RNA, which serves as a binding scaffold for the Cas nuclease.

Various gene editing technologies are known to those skilled in the art. Generally, gene editing systems employ editing polypeptides, which are proteins that function to edit a nucleobase, nucleotide, or nucleoside, typically using single-stranded or double-stranded DNA breaks. As used herein, the term “edit” refers to the insertion or deletion of basepairs (called “indels”) and the conversion of one nucleobase to another (e.g., A to G, A to C, A to T, C to T, C to G, C to A, G to A, G to C, G to T, T to A, T to C, T to G). Gene editors include, without limitation, homing endonucleases, zinc-finger nucleases (ZFNs), transcription activator-like effector (TALE) nucleases (TALENs), clustered regularly interspaced short palindromic repeats (CRISPR)-associated proteins (e.g., Cas9), and nucleobase editors of base editor systems. Homing endonucleases generally cleave their DNA substrates as dimers, and do not have distinct binding and cleavage domains. ZFNs recognize target sites that consist of two zinc-finger binding sites that flank a 5- to 7-base pair (bp) spacer sequence recognized by the FokI cleavage domain. TALENs recognize target sites that consist of two TALE DNA-binding sites that flank a 12- to 20-bp spacer sequence recognized by the FokI cleavage domain. In some cases, gene editing comprises CRISPR-targeted, TALEN-targeted, or ZFN-targeted silencing of genes via methylation. Such gene editing techniques employ targeted DNA methylation to silence specific genes without altering the host genomic sequence. See, e.g., Lei et al., Nature Communications volume 8, Article number: 16026 (2017).

In some cases, gene editing is performed using an RNA-guided nuclease such as a CRISPR-Cas system, such as a CRISPR-Cas9) system specific for the target gene (e.g., an immunosuppressive gene, a co-stimulatory molecule) that is disrupted. For CRISPR/Cas-based gene editing systems, the nucleobase editors are generally Cas polypeptides and variants thereof. Cas9 is a nuclease that targets to DNA sequences complementary to the targeting sequence within the single guide RNA (gRNA) located immediately upstream of a compatible protospacer adjacent motif (PAM) that may exist on either strand of the DNA helix. Examples of PAM sequence are known (see, e.g., Shah et al., RNA Biology 10 (5): 891-899, 2013).

When the gene editing system is a CRISPR/Cas system, the editing system is used in combination with one or more guide RNAs (gRNAs). For example, the CRISPR/Cas9 system uses an RNA-guide to target Cas9 nuclease to create a double stranded DNA break (DSB) at a specific location. These DSBs are repaired imperfectly, leading to indel formation, which disrupts gene expression. As used herein, a “guide RNA” (gRNA) is nucleotide sequence that is complementary to at least a portion of a target gene. In some embodiments, the sequence of PAM is dependent upon the species of Cas nuclease used in the architecture. It should be noted that the DNA-targeting sequence may or may not be 100% complementary to the target polynucleotide (e.g., gene) sequence. In certain embodiments, the DNA-targeting sequence is complementary to the target polynucleotide sequence over about 8-25 nucleotides (nts), about 12-22 nucleotides, about 14-20 nts, about 16-20 nts, about 18-20 nts, or about 8, 9, 10, 11, 12, 13, 14, 15, 16, 17, 18, 19, 20, 21, 22, 23, 24, or 25 nts. In certain embodiments, the complementary region comprises a continuous stretch of about 12-22 nts, preferably at the 3′ end of the DNA-targeting sequence. In certain embodiments, the 5′ end of the DNA-targeting sequence has up to 8 nucleotide mismatches with the target polynucleotide sequence. In certain embodiments, the DNA-binding sequence is about 50, 55, 60, 65, 70, 75, 80, 85, 90, 95, or 100% complementary to the target polynucleotide sequence. In some embodiments, the gRNA targeting sequences are specific for CISH, PD-1, or FAS and have the sequence SEQ ID NO: 7, 8, or 9, respectively.

In some embodiments, gene editing system components Cas9 and a guide RNA (gRNA) comprising a targeting domain, which targets a region of the genetic locus, are introduced into the cell. In some embodiments, the gene editing system components comprise a ribonucleoprotein (RNP) complex of a Cas9 polypeptide and a gRNA (Cas9/gRNA RNP).

It will be understood that cells edited by CRISPR-Cas systems as described herein are non-naturally occurring. Methods for introducing the CRISPR-Cas system in a cell are known in the art, and are further described herein elsewhere. The cell comprising the CRISPR-Cas system, or having the CRISPR-Cas system introduced, according to the invention comprises or is capable of expressing the individual components of the CRISPR-Cas system to establish a functional CRISPR complex, capable of modifying (such as cleaving) a target DNA sequence. Accordingly, as referred to herein, the cell comprising the CRISPR-Cas system can be a cell comprising the individual components of the CRISPR-Cas system to establish a functional CRISPR complex, capable of modifying (such as cleaving) a target DNA sequence. Alternatively, as referred to herein, and preferably, the cell comprising the CRISPR-Cas system can be a cell comprising one or more nucleic acid molecule encoding the individual components of the CRISPR-Cas system, which can be expressed in the cell to establish a functional CRISPR complex, capable of modifying (such as cleaving) a target DNA sequence.

Gene editing systems or components thereof (e.g., a nucleobase editor protein, a gRNA) are introduced into a cell (e.g., a γδ T cell) by methods known in the art. As used herein, the term “introducing” encompasses a variety of methods of introducing DNA or proteins into a cell, either in vitro or in vivo, such methods including transformation, transduction, transfection (e.g. electroporation), nucleofection (an electroporation-based transfection method which enables transfer of nucleic acids such as DNA and RNA into cells by applying a specific voltage and reagents), lipofection, and infection. Where the introducing involves electroporation (e.g., nucleofection), a polynucleotide (e.g., a plasmid, a single stranded DNA, a minicircle DNA, RNA) is electroporated into a target cell. Vectors are useful for introducing DNA encoding molecules into cells. Any appropriate delivery vector can be used with the methods described herein. For example, delivery vectors include exosomes, viruses (viral vectors), and viral particles. Preferably, the delivery vector is a viral vector, such as a lenti- or baculo- or preferably adeno-viral/adeno-associated viral (AAV) vectors, but other non-viral means of delivery are known (such as yeast systems, microvesicles, gene guns/means of attaching vectors to gold nanoparticles). Other methods of introducing a nucleic acid into a host cell are known in the art, and any known method can be used to introduce a nucleic acid (e.g., vector or expression construct) into a cell for the methods provided herein. Suitable methods include, include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv. Drug Deliv. Rev.), and the like.

In CRISPR/Cas based gene editing, a guide RNA and a site-directed modifying polypeptide (i.e., site-directed polypeptide) form a complex (i.e., bind via non-covalent interactions). The guide RNA provides target specificity to the complex by comprising a nucleotide sequence that is complementary to a sequence of a target DNA. The site-directed modifying polypeptide of the complex provides the site-specific activity. In other words, the site-directed modifying polypeptide is guided to a target DNA sequence (e.g., a target sequence in a chromosomal nucleic acid; a target sequence in an extrachromosomal nucleic acid, e.g., an episomal nucleic acid, a minicircle, etc.; a target sequence in a mitochondrial nucleic acid; a target sequence in a chloroplast nucleic acid; a target sequence in a plasmid; etc.) by virtue of its association with the protein-binding segment of the guide RNA. The site-directed modifying polypeptide, e.g., Cas, or in some embodiments Cas), catalyzes a break in the DNA strand.

As used herein, “non-homologous end joining (NHEJ)” is the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

Site-directed polypeptides can introduce double-strand breaks or single-strand breaks in nucleic acid, (e.g., genomic DNA). The double-strand break can stimulate a cell's endogenous DNA-repair pathways (e.g., homology-dependent repair (HDR) and non-homologous end joining (NHEJ) or alternative non-homologous end joining (A-NHEJ) or microhomology-mediated end joining (MMEJ)). NHEJ can repair cleaved target nucleic acid without the need for a homologous template. This can sometimes result in small deletions or insertions (indels) in the target nucleic acid at the site of cleavage and can lead to disruption or alteration of gene expression. HDR can occur when a homologous repair template, or donor, is available. The homologous donor template comprises sequences that are homologous to sequences flanking the target nucleic acid cleavage site. The sister chromatid is generally used by the cell as the repair template. However, for the purposes of genome editing, the repair template is often supplied as an exogenous nucleic acid, such as a plasmid, duplex oligonucleotide, single-strand oligonucleotide or viral nucleic acid. With exogenous donor templates it is common to introduce additional nucleic acid sequence (such as a transgene) or modification (such as a single base change or a deletion) between the flanking regions of homology so additional or altered nucleic acid sequence also becomes incorporated into the target locus. MMEJ results in a genetic outcome that is similar to NHEJ in that small deletions or insertions can occur at the cleavage site. MMEJ makes use of homologous sequences of a few base pairs flanking the cleavage site to drive a favored end-joining DNA repair outcome. In some instances, it may be possible to predict likely repair outcomes based on analysis of potential microhomologies in the nuclease target regions.

Thus, in some cases, homologous recombination is used to insert an exogenous polynucleotide sequence into the target nucleic acid cleavage site. An exogenous polynucleotide sequence is termed a donor polynucleotide herein. In some embodiments, the donor polynucleotide, a portion of the donor polynucleotide, a copy of the donor polynucleotide, or a portion of a copy of the donor polynucleotide is inserted into the target nucleic acid cleavage site. In some embodiments, the donor polynucleotide is an exogenous polynucleotide sequence, i.e., a sequence that does not naturally occur at the target nucleic acid cleavage site.

The modifications of the target DNA due to NHEJ and/or HDR can lead to, for example, mutations, deletions, alterations, integrations, gene correction, gene replacement, gene tagging, transgene insertion, nucleotide deletion, gene disruption, translocations and/or gene mutation. The processes of deleting genomic DNA and integrating non-native nucleic acid into genomic DNA are examples of genome editing.

As used herein, “transposon” refers to transposable elements (TEs), also known as “jumping genes,” which are DNA sequences that move from one location on the genome to another. “Transposon mutagenesis”, or “transposition mutagenesis”, is a biological process that allows genes to be transferred to a host organism's chromosome, interrupting or modifying the function of an extant gene on the chromosome and causing mutation. In some embodiments, the transposon is Snoke transposon. Snoke is a hyperactive Tc-Buster transposase that the inventors have used to insert the CAR-containing nanoplasmid transposon into the genomes of target cells. After recognizing transposon terminal repeat sequences within the nanoplasmid, the transposase (Snoke) integrates the CAR construct into the cell genome. Although it yields nonspecific integration, transposons represent a nonviral method of gene transfer, and the inventors have used it to consistently achieve>40% stable integration in gamma delta T cells. As used herein, “nanoplasmid” refers to small plasmid-like DNA molecules. More information regarding nanoplasmids may be found in U.S. Patent Publication No. US20150191735.

Methods and techniques for assessing the expression and/or levels of cell markers are known in the art. Antibodies and reagents for detection of such markers are well known in the art, and readily available. Assays and methods for detecting such markers include, but are not limited to, flow cytometry, including intracellular flow cytometry, ELISA, ELISPOT, cytometric bead array or other multiplex methods, Western Blot and other immunoaffinity-based methods. In some embodiments, the modified cells can be detected by flow cytometry or other immunoaffinity based method for expression of a marker unique to such cells, and then such cells can be co-stained for another marker.

As used herein, “FasL” refers to the protein “Fas ligand” which is a homotrimeric type II transmembrane protein expressed on cytotoxic T lymphocytes. It signals through trimerization of FasR, which spans the membrane of the “target” cell. This trimerization usually leads to apoptosis, or cell death. As used herein, “FasL blocking reagent” refers to any reagent known in the art that is capable of blocking or interrupting Fas from binding to FasL. By way of example, but not by way of limitation, FasL blocking reagents include antibodies, e.g., antibodies that are specific for FasL and prevent, or block, FasL ligation to FasR.

In another aspect of the current disclosure, kits for expanding polyclonal gamma delta T cells (GDTCs) are provided. In some embodiments, the kits comprise: i) anti-T cell receptor gamma delta antibody; ii) anti-CD28 antibody; and iii) one or more of IL-2, IL-7, and IL-15 cytokines. In some embodiments, the kits comprise IL-2. In some embodiments, the kits comprise IL-7. In some embodiments, the kits comprise IL-15. In some embodiments, the kits comprise IL-2 and IL-7. In some embodiments, the kits comprise IL-2 and IL-15. In some embodiments, the kits comprise IL-7 and IL-15. In some embodiments, the kits comprise IL-2, IL-7, and IL-15.

In another aspect, provided herein are methods for using the genetically modified in vitro expanded GDTCs described herein. For example, genetically modified GDTCs obtainable by the methods disclosed herein may be used for subsequent steps such as research, diagnostics, pharmacological or clinical applications known to the person skilled in the art.

In some cases, genetically modified GDTCs may be used to treat or prevent a disease or condition in a subject. In some cases, the method comprises introducing a nucleic acid encoding a chimeric antigen receptor (CAR) into a genetically modified γδ T cell, where the CAR has specificity for a surface antigen of a tumor cell and the ability to activate a T cell, expanding a culture of the genome-edited GDTCs ex vivo, and then administering the genome-edited GDTCs into a patient. Preferably, the genomeedited GDTCs are obtained according to the methods described herein. The disease could include, for example, cancer, a precancerous condition, infection with a pathogen (including, for example, malaria), or a viral infection. In some cases, the genetically modified GDTCs of this disclosure have an increased capacity to treat various cancer types including, without limitation, leukemia, neuroblastoma, and carcinomas, but are modified to reduce the likelihood of uncontrolled inflammation and associated unwanted tissue destruction which may be linked to γδ T-cell-based therapy.

In some embodiments, it is preferred that the cells are used for cancer immunotherapy. Advantageously, γδ T cell-mediated cytotoxicity does not rely on the presentation of self-human leukocyte antigens and they are not involved in graft-versus-host disease (GVHD). Accordingly, GDTCs of this disclosure have a high potential for off-the-shelf immunotherapies. In some cases, for example, GDTCs can be produced from healthy patients and given to patients whose immune systems are too compromised to be receptive to more conventional immunotherapies. Such allogenic immunotherapies are not limited by donor-matching.

In some cases, GDTCs genetically modified as described herein can be used to treat various conditions including cancer. For example, GDTCs obtained as described herein can be used to provide immunotherapy to a subject. Generally, the method comprises administering to a subject in need thereof a therapeutic composition comprising CAR-expressing GDTCs in which the antigen recognition region of the chimeric antigen receptor specifically binds to an antigen associated with the condition (e.g., particular cancer or tumor type). To “treat” a disease as the term is used herein, means to reduce the frequency or severity of at least one sign or symptom of a disease or disorder experienced by a subject. As used herein, the term “therapeutic” means a treatment or a therapeutic effect is obtained by suppression, remission, or eradication of a disease state.

In some cases, the condition is cancer or a precancerous condition. The cancer may include, for example, bone cancer, brain cancer, breast cancer, cervical cancer, cancer of the larynx, lung cancer, pancreatic cancer, prostate cancer, skin cancer, cancer of the spine, stomach cancer, uterine cancer, hematopoietic cancer, and/or lymphoid cancer, etc. A hematopoietic cancer and/or lymphoid cancer may include, for example, acute myelogenous leukemia (AML), acute lymphoblastic leukemia (ALL), myelodysplastic syndromes (MDS), non-Hodgkin lymphoma (NHL), chronic myelogenous leukemia (CML), Hodgkin's disease, and/or multiple myeloma. The cancer may be a metastatic cancer. The precancerous condition can be a preneoplastic lesion.

In some cases, the GDTCs are genetically modified ex vivo and contacted to an antigen, polypeptide, or peptide associated with various immunotherapies or gene therapy. In such cases, the modified cells are then returned to the subject as an autologous transplant in advance of the immunotherapy or gene therapy. As used herein, the term “autologous” is meant to refer to any material derived from the same individual to which it is later to be re-introduced into the individual.

In some cases, genetically modified GDTCs as described herein are provided to a subject in need thereof as a pharmaceutical composition comprising the modified cells and a pharmaceutically acceptable carrier. Carriers which may be used with the genetically modified GDTCs of the present invention will be well known to those of skill in the art. Methods for formulating the pharmaceutical composition and selecting appropriate doses are well known to those of skill in the art. An appropriate dosage of the pharmaceutical composition of the present invention may be variously prescribed depending on factors such as a formulation method, an administration manner, the age, body weight, sex, administration time and administration route of the patient. The dosage may also depend on the preparation method and yield.

In another aspect, provided herein are methods of targeting a tumor using genetically modified GDTCs. For example, a genome-edited GDTC may be administered to inhibit the growth of a tumor in a subject. In some embodiments, the tumor may include a solid tumor.

The genetically modified GDTCs and/or GDTC subsets can also be used as a pharmaceutical composition in the therapy, e.g., cellular therapy, or prevention of diseases. The pharmaceutical composition may be transplanted into an animal or human, preferentially a human patient. The pharmaceutical composition can be used for the treatment and/or prevention of diseases in mammals, especially humans, possibly including administration of a pharmaceutically effective amount of the pharmaceutical composition to the mammal. Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

The composition of genetically modified GDTCs obtained by the methods of this disclosure may be administered either alone, or as a pharmaceutical composition in combination with diluents and/or with other components such as cytokines or cell populations. Briefly, pharmaceutical compositions of the present invention may comprise the genome-edited GDTCs as described herein, in combination with one or more pharmaceutically or physiologically acceptable carriers, diluents, or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine: antioxidants; chelating agents such as EDTA or glutathione; adjuvants (e.g., aluminum hydroxide); and preservatives.

A genome-edited GDTC may be administered to a subject before, during, and/or after other treatments. Such combination therapy may involve administering genome-edited GDTCs before, during and/or after the use of other anti-cancer agents including, for example, a cytokine: a chemokine; a therapeutic antibody including, for example, a high affinity anti-CMV IgG antibody; an antioxidant; a chemotherapeutic agent; and/or radiation. The administration or preparation may be separated in time from the administration of other anti-cancer agents by hours, days, or even weeks. Additionally or alternatively, the administration or preparation may be combined with other biologically active agents or modalities such as, but not limited to, an antineoplastic agent, and non-drug therapies, such as, but not limited to, surgery.

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

The term “subject” is intended to include living organisms in which an immune response can be elicited or modulated (e.g., mammals). A “subject” or “patient,” as used therein, may be a human or non-human mammal. Non-human mammals include, for example, livestock and pets, such as ovine, bovine, equine, porcine, canine, feline, and murine animals. Accordingly, the term “subject” or “patient” as used herein means any mammalian patient or subject to which the genetically modified cells described herein can be administered. Preferably, the subject is human.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The phrases “% sequence identity,” “percent identity,” or “% identity” refer to the percentage of amino acid residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other compositions described herein may be purified. As used herein, “purified” means separate from the majority of other compounds or entities and encompasses partially purified or substantially purified. Purity may be denoted by a weight by weight measure and may be determined using a variety of analytical techniques such as but not limited to mass spectrometry, HPLC, etc.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid.” “DNA,” “RNA,” and/or similar terms include nucleic acid analogs, i.e., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “hybridization” as used herein, refers to the formation of a duplex structure by two single-stranded nucleic acids due to complementary base pairing. Hybridization can occur between fully complementary nucleic acid strands or between “substantially complementary.” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions: the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, New York; Wetmur, 1991, Critical Review in Biochem. and Mol. Biol. 26 (3/4): 227-259; and Owczarzy et al., 2008, Biochemistry, 47:5336-5353, which are incorporated herein by reference).

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including.” “comprising.” or “having.” and variations thereof, is meant to encompass the elements listed thereafter and equivalents thereof, as well as additional elements. Embodiments recited as “including.” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

While the claims provided herein are directed to methods of treating a subject, both human and non-human subjects are envisioned. In addition, use of the compositions provided herein as medicaments for uses in therapy or for treating disease are also provided herein. Use of the compositions provided herein in the manufacture of a medicament for the treatment of a disease or condition are also encompassed.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### EXAMPLES

The following Examples are illustrative and should not be interpreted to limit the scope of the claimed subject matter.

### Example 1—Large-Scale Expansion of Engineered Human Gamma Delta T Cells

Methods: Gamma-delta T cell stimulation with plate-bound Pan-GDTCR/OKT3+soluble CD28:

Reagents: Pan gamma delta antibody (for plate bound stim): TCRγ/δ Antibody, anti-human, REAfinity™. www.miltenyibiotec.com/US-en/products/tcrg-d-antibody-anti-human-reafinity-rea591.html#pure: 100-ug-in-100-ul. Soluble CD28: CD28 Monoclonal Antibody (CD28.2), Functional Grade, eBioscience™ www.thermofisher.com/antibody/product/CD28-Antibody-clone-CD28-2-Monoclonal/16-0289-81.

### GDTC Base Media:

| Amount | | Units | Component | Vendor |
|---|---|---|---|---|
| 500 | 200 | | | |
| 427 | 170.8 | mL | OpTimizer T-cell | Gibco A10221-01 |
| | | | expansion medium | |
| 13 | 5.2 | mL | OpTimizer T-cell | Gibco A10484-01 |
| | | | Expansion Supplement | |
| 50 | 20 | mL | Valley Human AB serum | Gibco A25961-01 |
| 5 | 2 | mL | L-Glutamine | Gibco 25030-081 |

GDTC Complete Media. For Each mL of GDTC Base Media, Add:

| | | | Volume | | | |
|---|---|---|---|---|---|---|
| | Stock | | per 1 mL | Final | | |
| Component | Concentration | | media | Concentration | | Vendor |
| Recombinant Human IL-2 | 1000 | IU/uL | 1 uL | 1000 | IU/mL | Peprotech 200-02 |
| Recombinant Human IL-7 | 5 | ng/uL | 1 uL | 5 | ng/mL | Peprotech 200-07 |
| Recombinant Human IL-15 | 5 | ng/uL | 1 uL | 5 | ng/mL | Peprotech 200-15 |
| Penicillin-Streptomycin | 100X* | | 10 uL | 1X | | Millipore TMS-AB2-C |
| (Pen/Strep) | | | | | | |

*Note:
100X Pen/Strep contains 10,000 Units Penicillin and 10,000 μg Streptomycin per mL.

### Protocols:

### Initial Stim (Day 0):

Identify desired number of post-isolation GDTCs:
a. If greater than or equal to 1×10⁶ cells, stim in 24-well plate (1-1.5×10⁶ cells/well)
Use 250 uL antibody solution per well
b. If less than 1×10⁵ cells, use 48-well plate (3-5×10⁵ cells/well)
Use 200 uL antibody solution per well
Prepare antibody solution (on ice):
c. Composition: 10 ug antibody per mL PBS
d. Prepare 5% extra solution to account for pipetting error
e. Mix well by inverting/flicking or pipetting up-and-down
Add antibody solution to desired wells:
f. 250 uL/well in a 24-well plate; 200 μL/well in a 48-well plate
g. Surround stimmed wells with a ring of PBS (if possible)
2 mL PBS/well in a 24-well plate
1 mL PBS/well in a 48-well plate
Incubate plate (2 options):
h. Incubate plate overnight in a 4° C. refrigerator→ (Day-1)
Use when isolating GDTCs on the following day
i. Incubate plate for 2-2.5 hours in a 35° C. incubator→ (Day 0)
Use when isolating GDTCs immediately afterwards
Note: Do not incubate at 35° C. overnight, and preferably not for more than 2.5 hours. If the isolation is not complete within 2.5 hours, transfer the plate to a 4° C. refrigerator until the isolation is complete.
Isolate GDTCs:
j. Thaw PBMCs
Protocol→Takes 40-80 minutes, depending on number of vials
k. Isolate GDTCs using EasySep™ Human Gamma/Delta T Cell Isolation Kit (StemCell Technologies)
Protocol→Takes roughly 70 minutes
1. Spin down isolated GDTCs, resuspend in complete stim media
Final Media=GDTC complete media+2 μg/mL soluble CD28
1-1.5×10⁶ cells/mL for a 24-well plate; 0.6-1×10⁶ cells/mL for a 48-well plate Plate GDTCs:
m. Remove plate from incubator, then wash each well three times with PBS i. For each wash: Tilt the plate 30-45°, remove the solution currently in each well, set the plate back down, then gently pipette back in an equivalent amount of PBS. Remove all PBS after the final wash.
n. Plate cells at 1 mL/well (at 1-1.5×10⁶ cells/mL) for a 24-well plate
o. Plate cells at 0.5 mL/well (at 0.6-1×10⁶ cells/mL) for a 48-well plate
Place plate in incubator (35° C., 5% CO₂); initiate further experimentation
p. Note: For any passages or media changes performed over the course of the first ten days, do not use media containing CD28. Instead, allow the current CD28 to become more dilute over time (or be lost altogether following any pelleting events), since continuously high exposure levels can be harmful to the cells.
Re-Stim (Day 11 Post-Initial Stim):
1. Identify desired number of re-stimmed GDTCs:
If 1-1.5×10⁶, use 1 well of a 24-well plate
If 1.6-2.5×10⁶, use 1 well of a 12-well plate
If 2.6×10⁶-4.5×10⁶, use a 1 well of a 6-well plate
If 4.6×10⁶-10×10⁶, use an upright T-25
If >10×10⁶, use a flat T-25
Note: For large numbers of cells, it is possible to use multiple small wells instead of one large one (i.e. 2×24 instead of 1×12 for 2e6 cells). However, this is generally undesirable because stochastic re-stim responses can induce phenotypic discrepancies between the wells.
2. Prepare antibody solution (on ice):
Composition: 10 ug antibody per mL PBS
a. Prepare 5% extra solution to account for pipetting error
b Mix well by inverting/flicking or pipetting up-and-down
3. Add antibody solution to desired wells:
Scale solution volume to 250 uL/1.9 cm²
24-Well Plate: SA=1.9 cm²→250 uL/well
12-Well Plate: SA=3.8 cm²→500 μL/well
6-Well Plate: SA=9.5 cm²→1.25 mL/well
Upright T-25: SA=16 cm²→2.1 mL/flask
Flat T-25: SA=25 cm²→3.25 mL/flask
4. Incubate plate/flask (2 options):
a. Incubate overnight in a 4° C. refrigerator→ (Day 10)
Use when re-stimming GDTCs on the following day
b. Incubate for 2-2.5 hours in a 35° C. incubator→ (Day 11)
Use when re-stimming GDTCs on the same day
Do not incubate at 35° C. overnight, and preferably not for more than 2.5 hours. If the isolation is not complete within 2.5 hours, transfer the plate to a 4° C. refrigerator until the isolation is complete.
5. Re-Stim GDTCs:
a. Remove plate/flask from incubator, then wash each well three times with PBS
For each wash: Tilt the plate/flask 30-45°, remove the solution currently in each well, set the plate back down, then gently pipette back in an equivalent amount of PBS. Remove all PBS after the final wash.
b. Transfer the desired number of cells into each stim plate/flask
c. Bring each well up to optimal media volume using GDTC complete media
1 mL media (1-1.5×10⁶ cells/mL) for a 24-well plate (1.5 mL MAX)
1.5-2.5 mL media (1-1.5×10⁶ cells/mL) for a 12-well plate (3 mL MAX)
2.5-4 mL media (1-1.5×10⁶ cells/mL) for a 6-well plate (5 mL MAX)
4.5-7.5 mL media (1-2×10⁶ cells/mL) for a T-25 (8 mL MAX)
Note: In cases where the pre-transfer GDTCs are being cultured at low density (1×10⁶ cells mL), spin down the cells for 5 min at 400×g, then resuspend in GDTC complete media at optimal density before transferring to the stim plate flask.
d. Stim each well with 2 μg/mL soluble CD28-Gently swirl the plate/flask to facilitate diffusion.
6. Place plate in incubator (35° C., 5% CO₂); initiate further experimentation
Note: For any subsequent passages or media changes performed over the course of the experiment, do not use media containing CD28. Instead, allow the current CD28 to become more dilute over time (or be lost altogether following any pelleting events), since continuously high exposure levels can be harmful to the cells.
Results: Efficient Polyclonal expansion of GDTCs

### GDTC-23: Engineering Zo Stimulated GDTCs

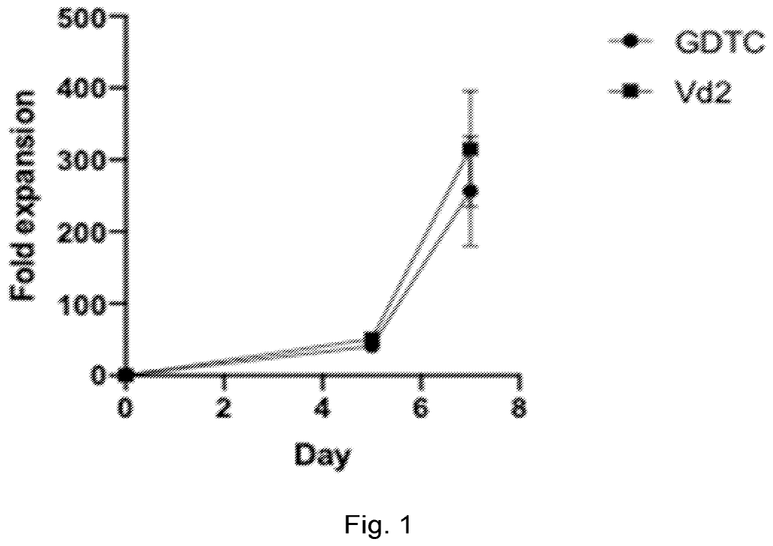

Day 7 Fold expansion (FIG. 1):

Significance: Evaluates fold expansion between days 0 and 7 post-Zoledronate (Zo) stim (FIG. 1). LP5→64.7×10⁶ Vd2 From initial GDTC 333-fold From initial Vd2 396-fold. LP6→51.3×10⁶ Vd2. From initial GDTC 180-fold. From initial Vd2 234-fold.

### GDTC-24: Optimizing Electroporation (EP or Zaps) for Zo Stimulated Cells

Day 4 EP cells Electroporated cells are also referred to as “zaps” and refers to cells that have been exposed to electroporation for the purpose of genetic modification. The terms can be used herein interchangeably. EP stands for electroporation or electroporated depending on context as used herein. (Day 7 Post-EP):

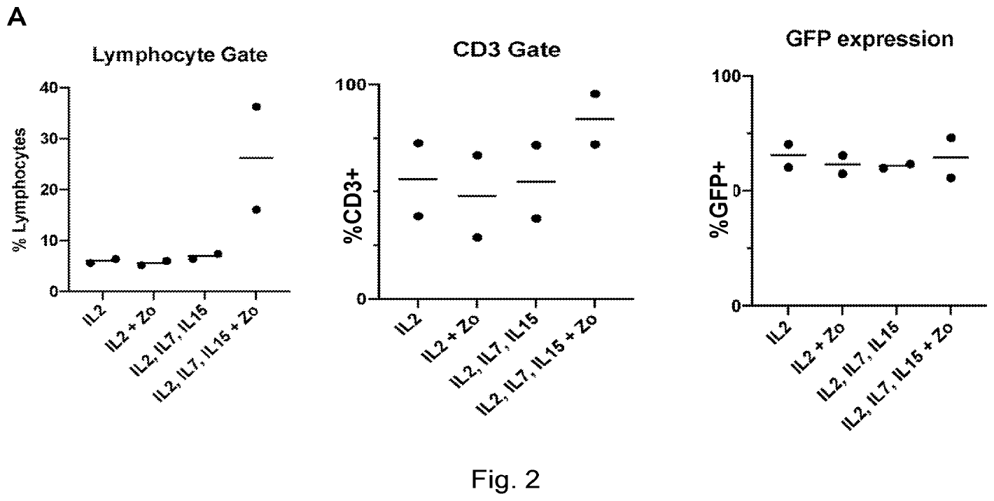

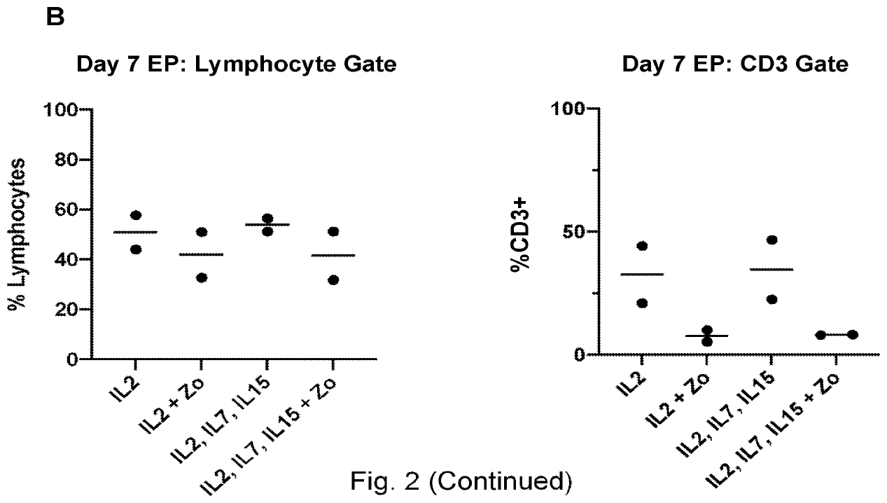

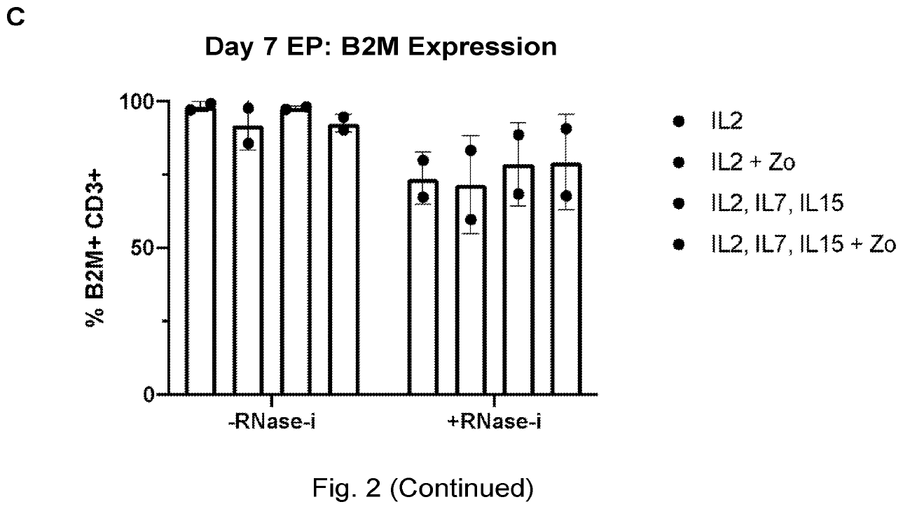

Significance: Evaluates % Lymphocytes, % CD3+, and % GFP+ for day 4 EP placed in recovery media containing: IL2 only; IL2+Zo; IL2, IL7, and IL15; and IL2, IL7, and IL15+Zo. Although all samples had roughly the same GFP positivity, “All Cytokines+Zo” had a significantly higher % Lymphocytes and % CD3+ (FIG. 2A). B2M knockout was included in these experiments because the inventors have seen successful Cas9-mediated editing with B2M guides in other cell lines, and it allows an easy readout using flow cytometry. It stands in contrast to the GFP pulse electroporations, which do not induce edits in the genome. The inventors sought to deduce whether any of the cytokine combinations would yield a significant difference in transfection efficiency; as expected, they do not. Instead, the greatest difference is seen in T cell expansion (% CD3+). In addition, this was an experiment where the inventors thought fratricide may have occurred in the B2M (beta 2 microglobulin) KO conditions.

### Day 7 EPs (Day 7 Post-EP):

Significance: Evaluates % Lymphocytes and % CD3+ for day 7 electroporated cells plated in the same recovery media conditions listed above. Demonstrates that for day 7 zaps, additional zoledronate exposure is deleterious to growth (FIG. 2B).

### Day 7 EP→B2M Expression

Significance: Evaluates % B2M+ for day 7 electroporated cells performed +/− RNase inhibitor (RNase-I) plated in the recovery media conditions listed above. Demonstrates the usefulness of RNase-I for increasing transfection efficiency (FIG. 2C).

### GDTC-26: Optimization of Scaled-Up Neon Electroporation for Zo Stimulated GDTCs

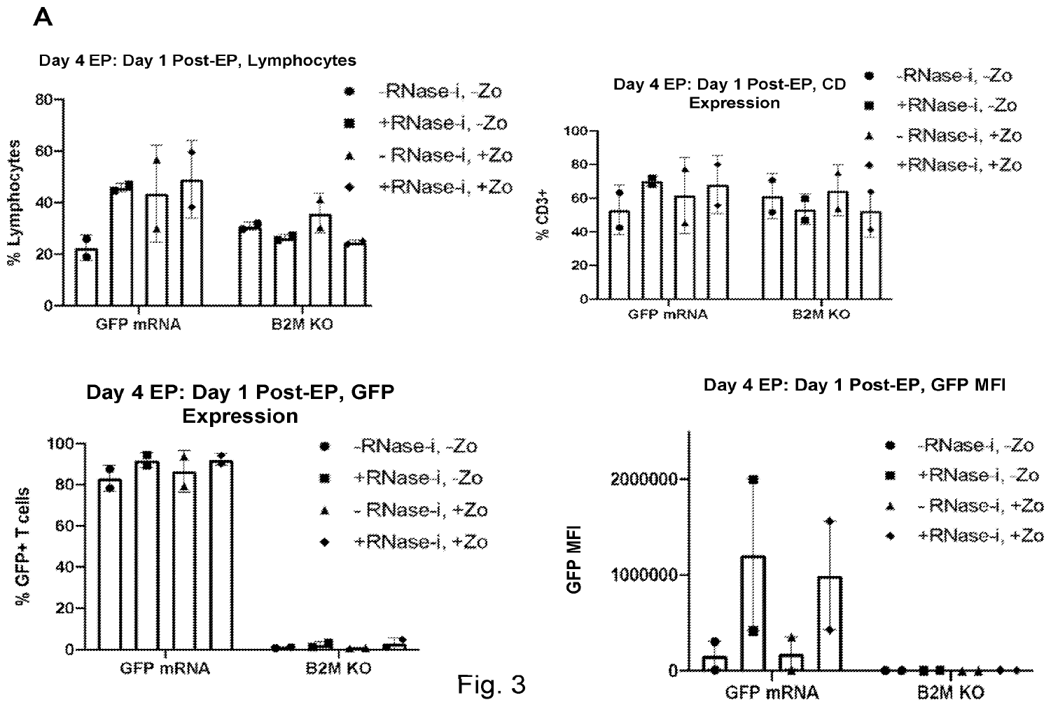

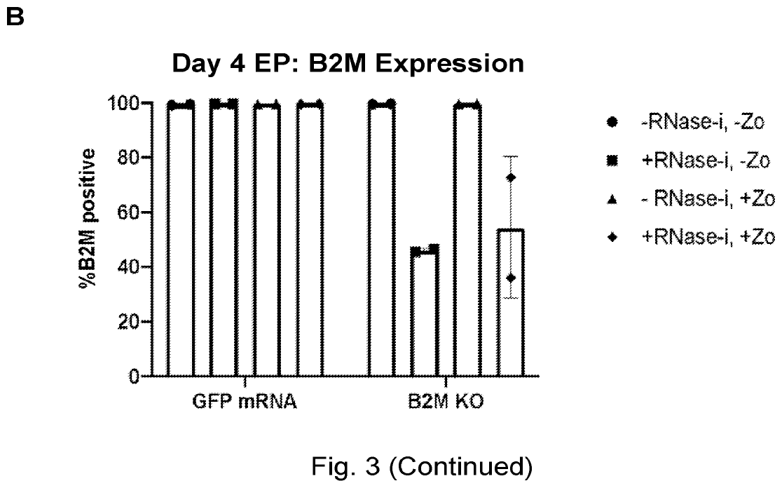

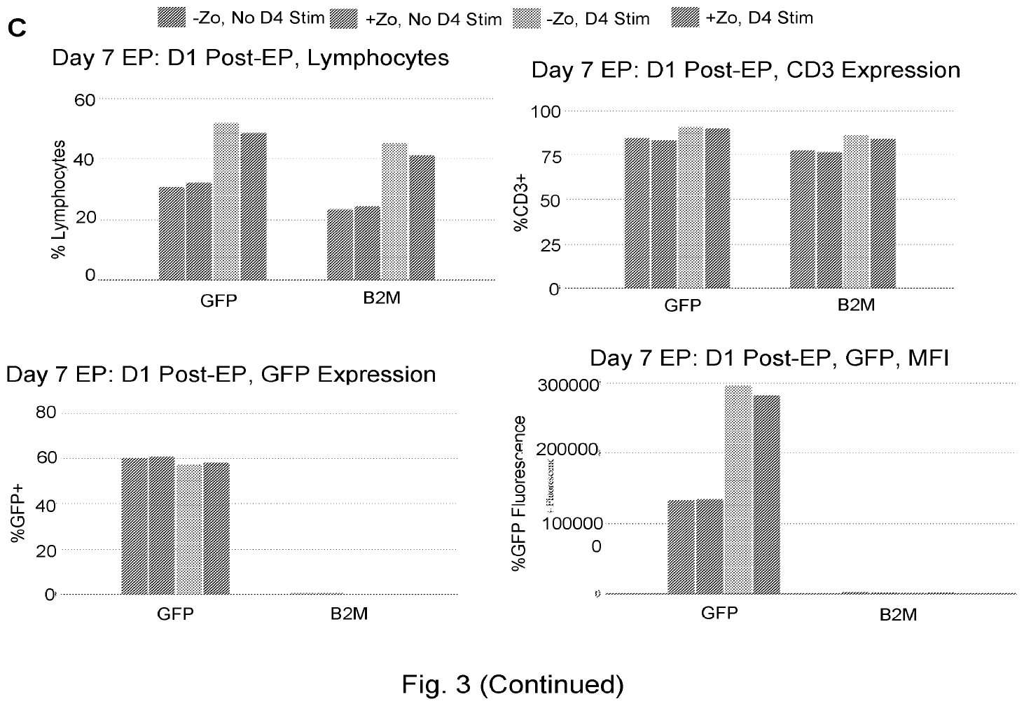

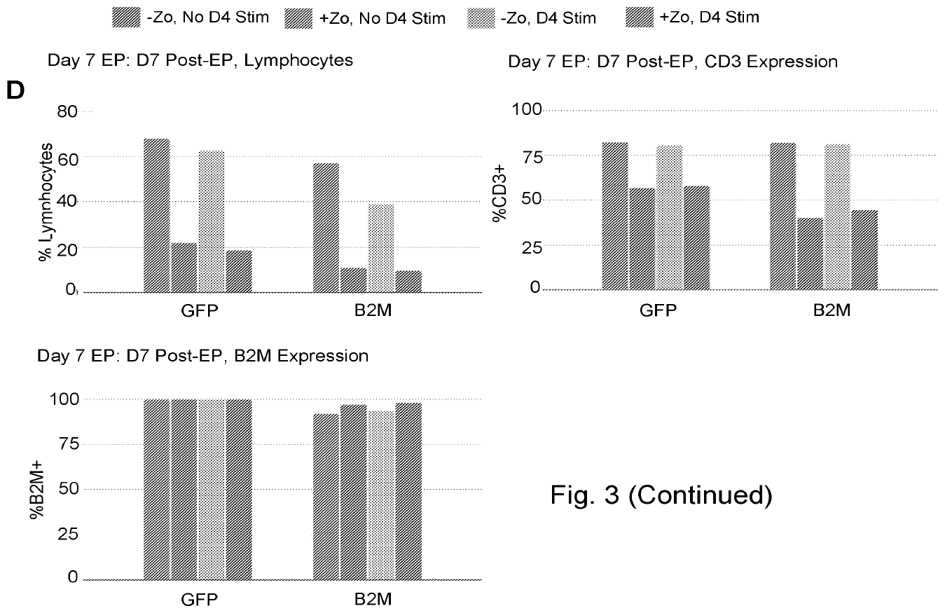

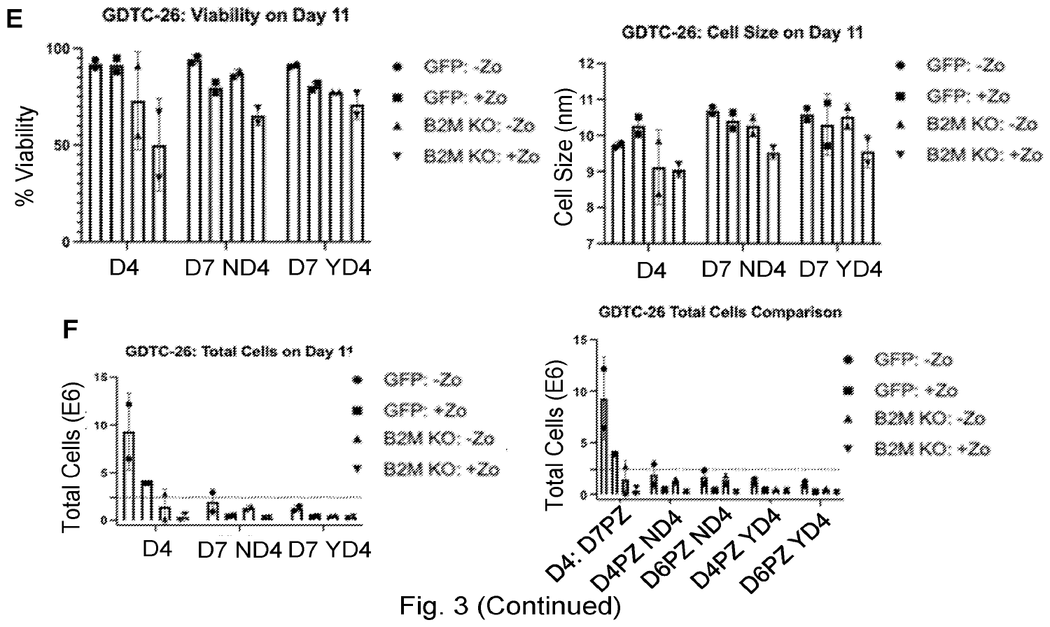

### Day 4 EP (Day 1 Post-EP)→Flow Data (FIG. 3A):

As used herein, “Neon” refers to the Neon Transfection System (www.thermofisher.com/order/catalog/product/MPK5000 #/MPK5000). This is the system the inventors used for all subsequent electroporations because it provided a higher transfection efficiency than other electroporation methods with less toxicity. Thus, “neon zaps” or “neon EP” refers to cells that have been electroporated with the neon system.

Significance: Evaluates % Lymphocytes, % CD3+, % GFP+, and GFP fluorescence intensity 1 day post-EP in recovery media containing +/− RNase-I and +/−Zo.

% GFP+ is >90% for both + and − RNase-I samples; however, GFP fluorescence intensity is significantly higher in the + RNase-I samples, reinforcing the utility associated with its use, and demonstrating that it could help improve knockout efficiency.

Lymphocyte % and CD3+% were higher in + RNase-I samples for GFP, but higher in-RNase-I samples for B2M KO (although the difference is likely not statistically significant). This could potentially provide evidence of Cas9-induced cell death, since the higher transfection efficiency induced by the RNase-I (see fluorescence intensity graph for GFP) would likely result in greater Cas9 activity in the relevant samples, producing lower lymphocyte and CD3 counts as a result of cell death (FIG. 3A).

### Day 4 EP Cells (Day 7 Post-EP)→Flow Data (FIG. 3B):

Significance: Evaluates % B2M+ for day 4 electroporated cells at day 7 post-EP in the recovery media conditions listed above. Demonstrates that RNase-I significantly improves B2M KO in GDTCs. B2M knockout calculated at an average of 50-55%, although fratricide could potentially have interfered with this data (see count data described below, FIG. 3B).

Day 7 EP cells (Day 1 Post-EP)→Flow Data (FIG. 3C):

Significance: Evaluates % Lymphocytes, % CD3+, % GFP+, and GFP fluorescence intensity 1-day post-zap for control and day 4 re-stimmed samples seeded in recovery media containing Rnase-I+/−Zo. It appears as though % lymphocytes (along with GFP fluorescence intensity) are significantly higher in D4 re-stimmed samples (FIG. 3C), although this trend does not continue for the duration of the experiment (see next set of figures).

Day 7 EP cells (Day 7 Post-EP)→Flow Data (FIG. 3D): Significance: Evaluates % Lymphocytes, % CD3+, % GFP+, and GFP fluorescence intensity 7 days post-EP for control and day 4 re-stimmed samples seeded in recovery media containing Rnase-I+/−Zo.

It appears that by day 14, culture health is influenced more by the presence or absence of Zo in the recovery media (−Zo is better) than by whether or not the samples received a re-stim on day 4 (FIG. 3D). B2M knockout was fairly limited in general, although the strength of this figure's conclusions are limited by the possibility of fratricide

### Day 4/7 EP Cell Size and Viability at Day 11 Overall (FIG. 3E):

Significance: Provides a consistent time point for evaluating cell size and viability in day 4 and day 7 zaps placed in recovery media containing Rnase-I+/−Zo. The most significant conclusion is that both viability and cell size are generally higher in media without Zo for the day 7 EP cells, indicating healthier cultures within the relevant samples. The conclusions for D4 are a bit more difficult to parse out, since it appears that at least one donor crashed in the B2M KO conditions (FIG. 3E). Relatively low cell size in “D4 GFP: −Zo” could potentially be correlated with the higher relative expansion observed for this condition (see next section).

### Day 4/7 Total Cells (FIG. 3F):

Significance: Evaluates total cell counts for the aforementioned conditions at fixed points in time. Each graph below demonstrates a different iteration of the data: the first only demonstrates data from day 11 overall (day 7 post-EP for the day 4 EP cells, and day 4 post-EP for the +/− day 4 re-stimmed day 7 EP cells), while the second includes additional data from day 6 post-EP for the day 7 EP cells. Suggests that the optimal strategy for EPing Zo-stimmed GDTCs is to perform day 4 zaps without Zo in the recovery media.

### GDTC-28: Optimization of EP Intensity for Zo Stimulated GDTC

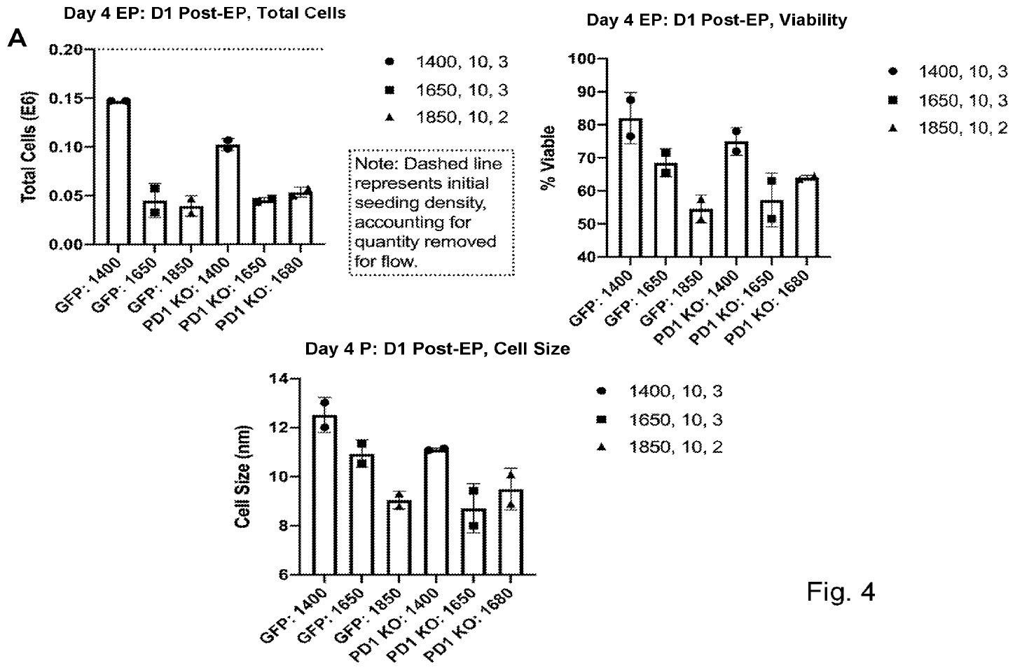

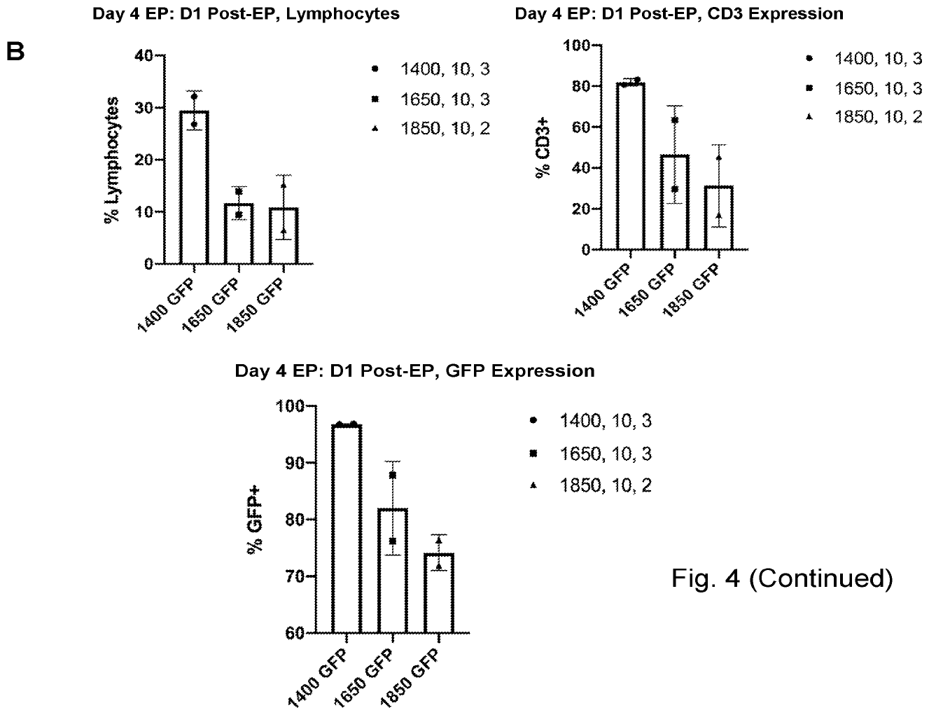

### Day 4 EP Cells→Count Data: (FIG. 4A)

Significance: Evaluates total cell number, average cell size, and viability 1 day post-EP for day 4-EPed GFP and PD1 KO GDTCs using various EP intensities. Demonstrates that increased EP intensity results in decreased culture health and expansion; greater amounts of voltage are significantly harsher on the cells.

Day 4 EP cells→Flow Data (FIG. 4B):

Significance: Evaluates % Lymphocytes, % CD3+, and % GFP+1 day post-EP for the day 4 EP cells described above. Corroborates observation that increased EP intensity reduces culture health (evidenced by lower lymphocyte and CD3+ percentages). Also demonstrates that increased EP intensity does not produce any increase in transfection efficiency (GFP positivity is actually lower in these samples), supporting the continued use of 1400 volt EP condition in future experiments.

### Plate-Stimulation Experiments:

### GDTC-30:

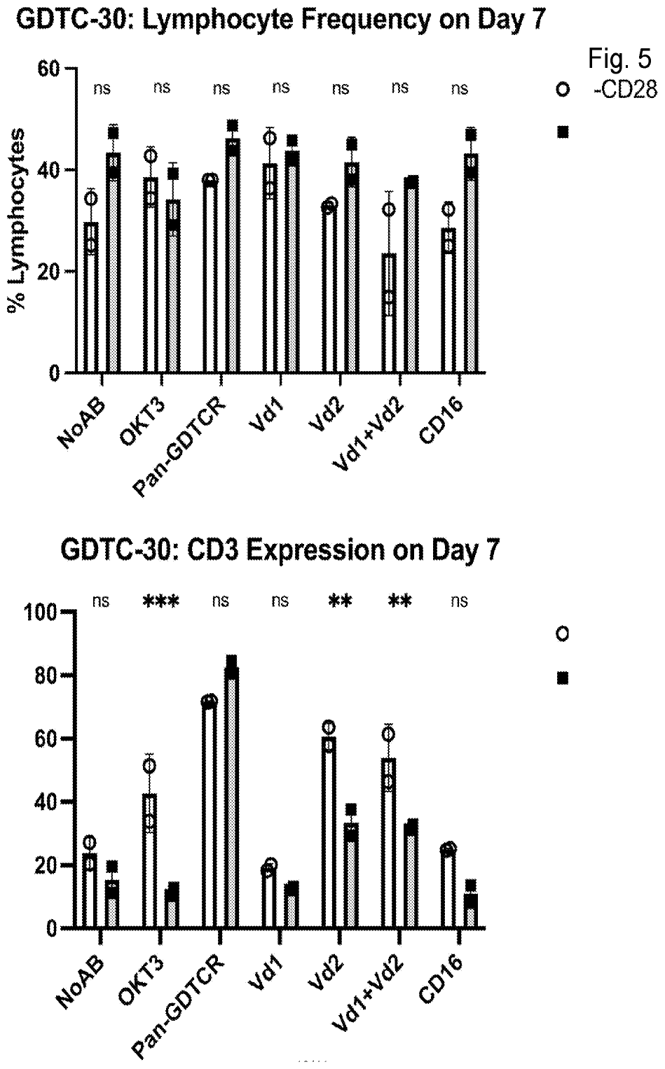

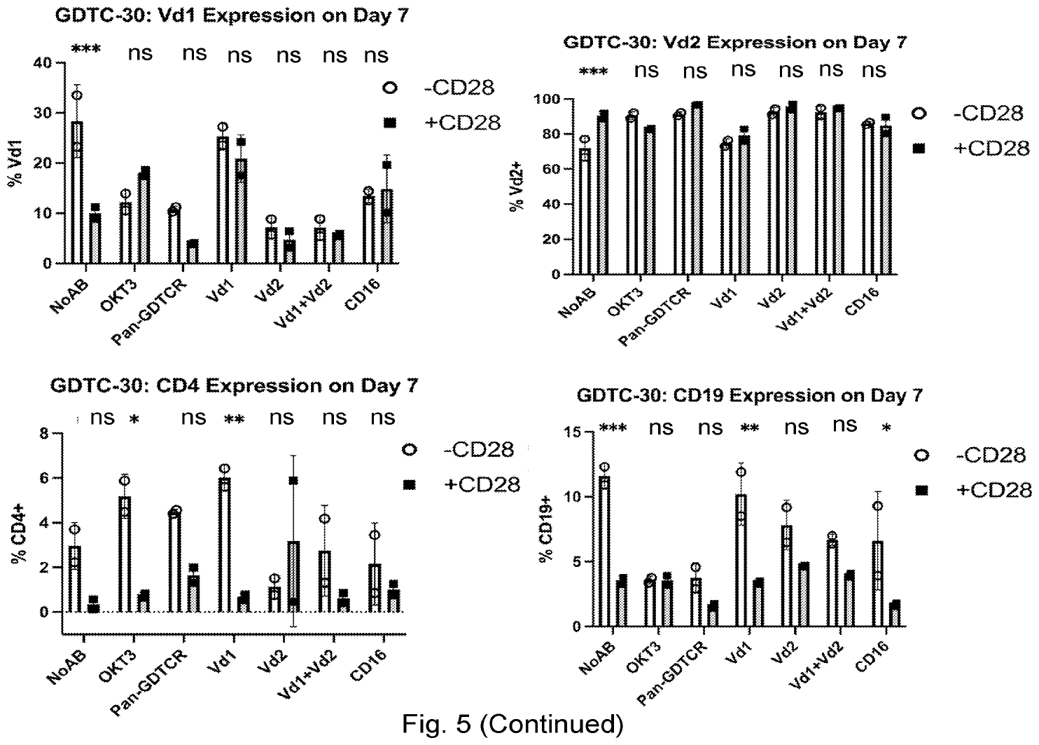

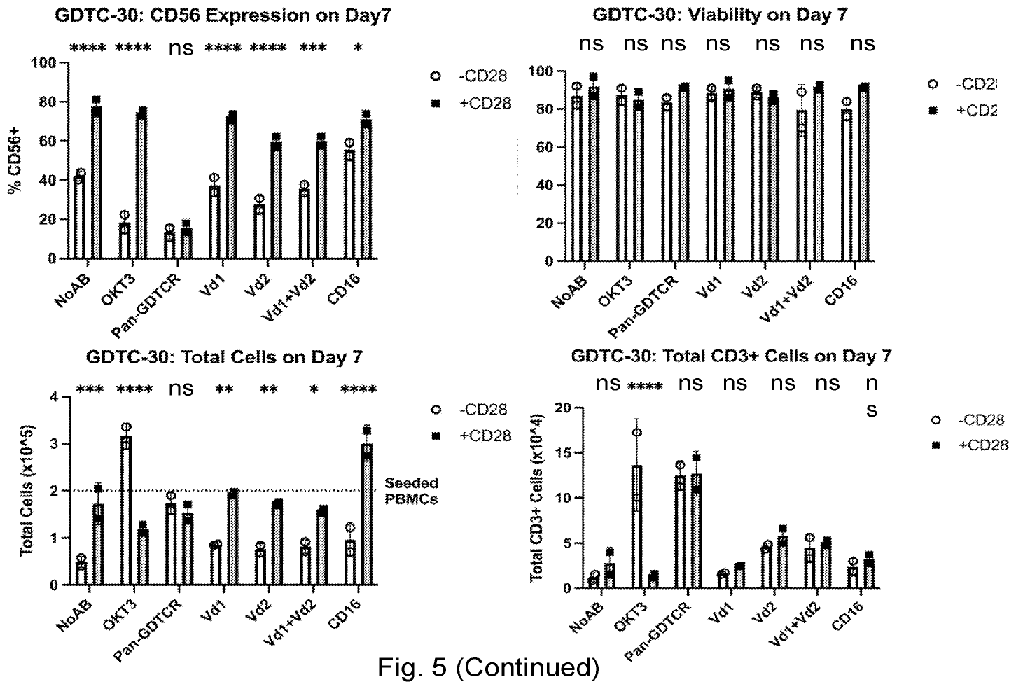

### Plate-Stim Comparison→Day 7 Count/Flow Data (FIG. 5):

Significance: Informs future use of pan-GDTCR antibody for plate-stim procedures, given that it produced the highest CD3 positivity (without any relative increase in CD4 positivity) and one of the highest total CD3+ counts (along with OKT3, which we were planning on trying out anyways).

Notes: All antibody concentrations were at 1/50th of the desired amount, but the inventors decided that the trends reflected in the data were likely to remain the same regardless of concentration. Graphed viability comes from count data, not flow cytometry. Error bars represent +/− one standard deviation. Statistical analysis comes from a multiple comparisons test as part of a 2-way ANOVA. In each case, the significance reflects the impact of +/−CD28 on the dependent variable for each plate-stim condition. Gating: Lymphocytes, Single cells, Viable, CD3, Vd1, Vd2, CD4, CD19, CD56.

### GDTC-31:

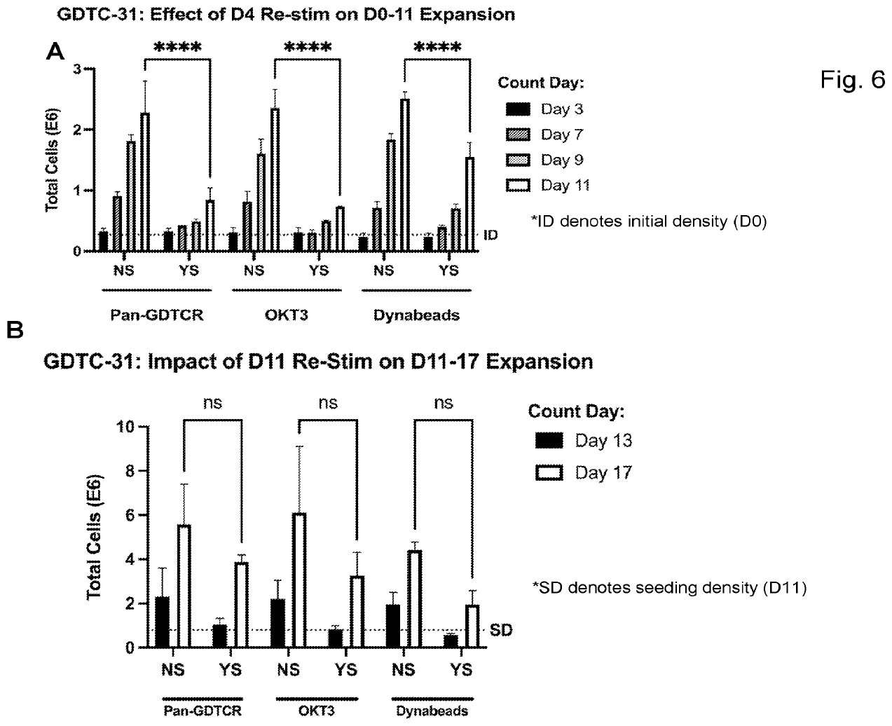

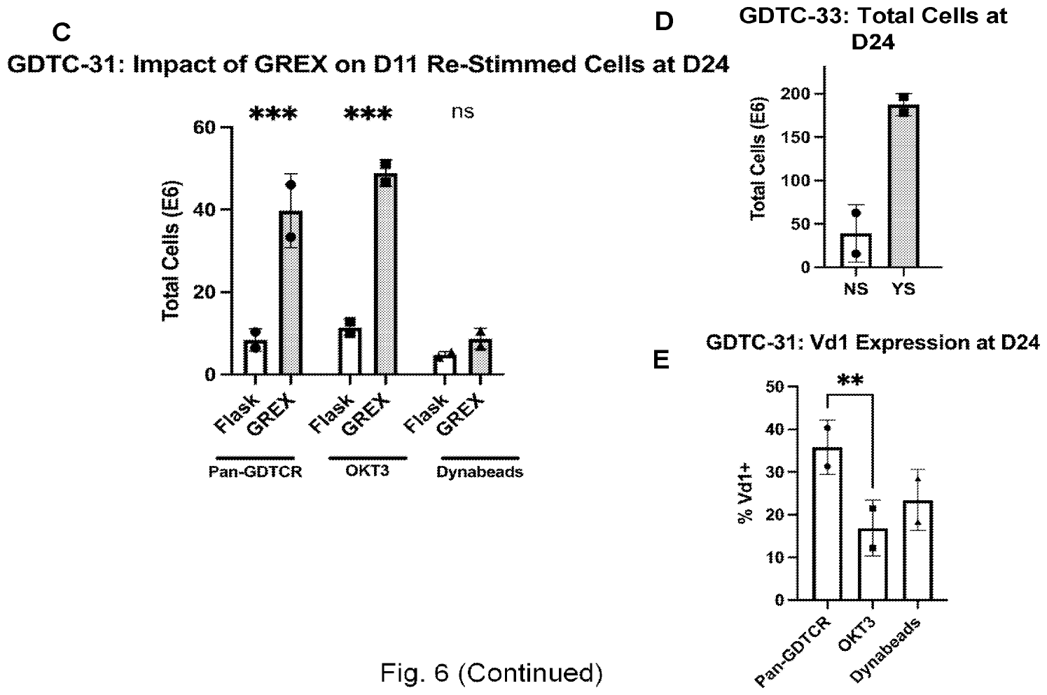

### +/− Day 4 Re-Stim Count Data (DO-11) (FIG. 6A):

Significance: Represents a preliminary expansion curve for our three main stim conditions of interest: pan-GDTCR+CD28, OKT3+CD28, and Dynabeads. Also demonstrates the deleterious effects of a D4 re-stim, which we hypothesize is far too early.

Notes: “ID denotes initial density” is intentionally placed in the corner so that it can be cropped out later; in a publication, this information would likely appear in our figure legend instead of the figure itself. Asterisks indicate the statistical significance of the impact of a day 4 re-stim on total cells at D11. Values represent multiple comparisons derived from a standard two-way ANOVA.NS=“No Re-Stim,” YS=“Yes Re-Stim”. Error bars are one standard deviation above the mean.

### +/− Day 11 Re-Stim Count Data (FIG. 6B):

Significance: Demonstrates superiority of the day 11 re-stim, since—unlike the day 4 re-stim—it did not produce a statistically significant difference in expansion over the course of the following week.

Notes: Asterisks indicate a non-statistically significant difference in expansion for all three stim conditions as a function of +/−D11 re-stim. Values represent multiple comparisons derived from a standard two-way ANOVA. Error bars are one standard deviation above the mean.

### +/− GREX for D11 Re-Stimulated Cells (FIG. 6C):

As used herein, “GREX” or “G-Rex” refers to flasks designed for the expansion of hematopoietic cells, e.g. GDTCs. More information regarding GREX flasks can be found at Bajgain, P. et al. “Optimizing the production of suspension cells using the G-Rex “M” series”. Methods and Clinical Development. Vol. 1, 2014, which is incorporated by reference herein in its entirety.

Significance: Informs our future culture workflow, since we ended up deciding that D11 re-stim→48 hr wait→transfer to GREX yielded optimal expansion. Demonstrates superiority of GREXs for long-term GDTC culture.

Notes: Asterisks indicate statistically significant impact of GREX use on expansion for Pan-GDTCR and OKT3. Values represent multiple comparisons derived from a standard two-way ANOVA. Error bars are one standard deviation above and below the mean. Dynabeads were removed during transfer to GREX, so all three re-stims were terminated at the established 48-hour timepoint.

### Vd1 Expression at Day 24 (FIG. 6D):

Significance: Demonstrates that pan-GDTCR stimulation results in significantly higher phenotypic heterogeneity than OKT3 (the other potential plate-stim option), as evidenced by a substantial increase in Vd1 expression.

Notes: Asterisks indicate a statistically significant difference in Vd1 expression on day 24 between samples stimmed with pan-GDTCR and OKT3. Values derived from a paired t-test (pan-GDTCR vs Dynabeads) with a Bonferroni correction (alpha: 0.05→0.0133; p-value of 0.0050 is still lower than alpha). Error bars are one standard deviation above and below the mean. Regardless of stim condition, one donor (LP8) had a consistently higher level of Vd1 expression than the other (LP7); the upper dot is always LP8, while the lower one is LP7.

### GDTC-32:

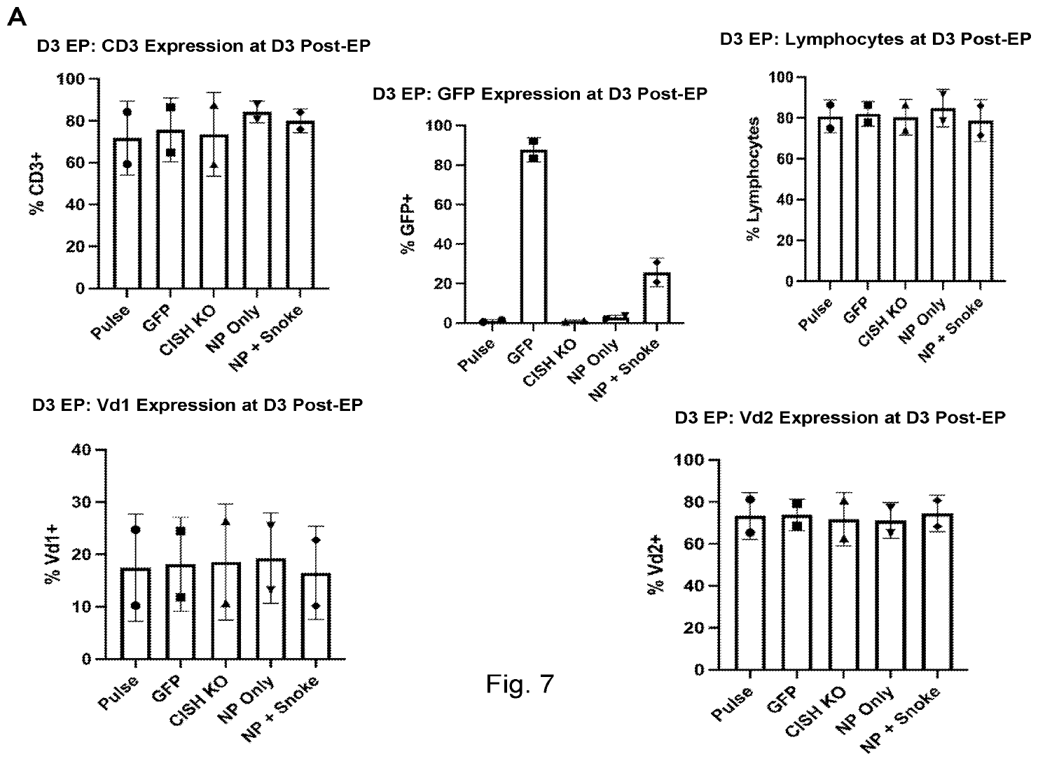

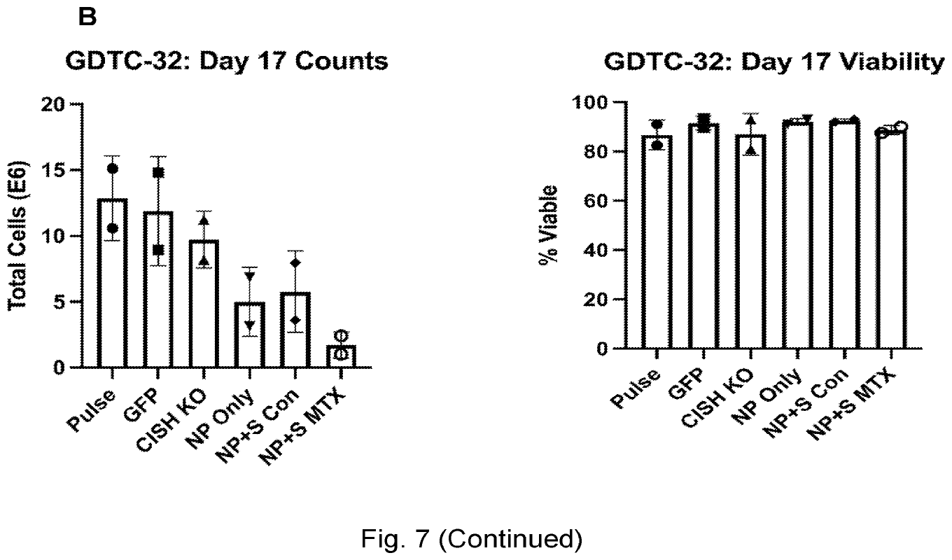

### Day 3 Post-EP Flow Data (FIG. 7A):

Significance: Illustrates flow phenotype and GFP positivity for day 3 EP cells. Notes: Error bars are one standard deviation above and below the mean. Donors are LP7 (lower Vd1) and LP8 (higher Vd1). Gating: Lymphocytes, Single Cells, Viable, GFP+, CD3+, Vd1+, Vd2+ Day 17 Count Data (FIG. 7B):

Significance: Illustrates the final cell total and viability for GDTC-33.

Notes: Error bars are one standard deviation above and below the mean. MTX=methotrexate; selection took place on day 6. Initial seeding density was ˜280k cells for all conditions.

### GDTC-33:

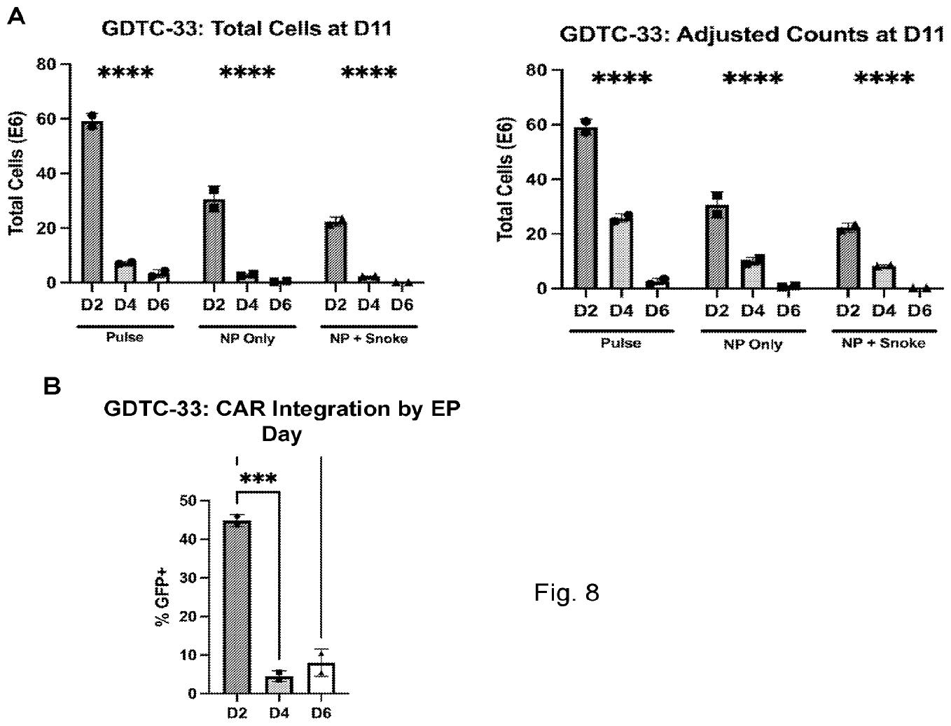

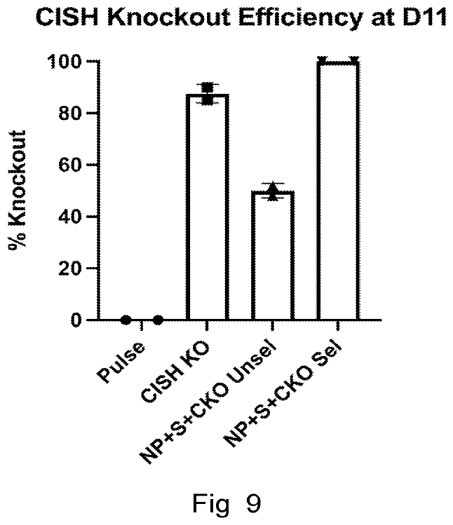

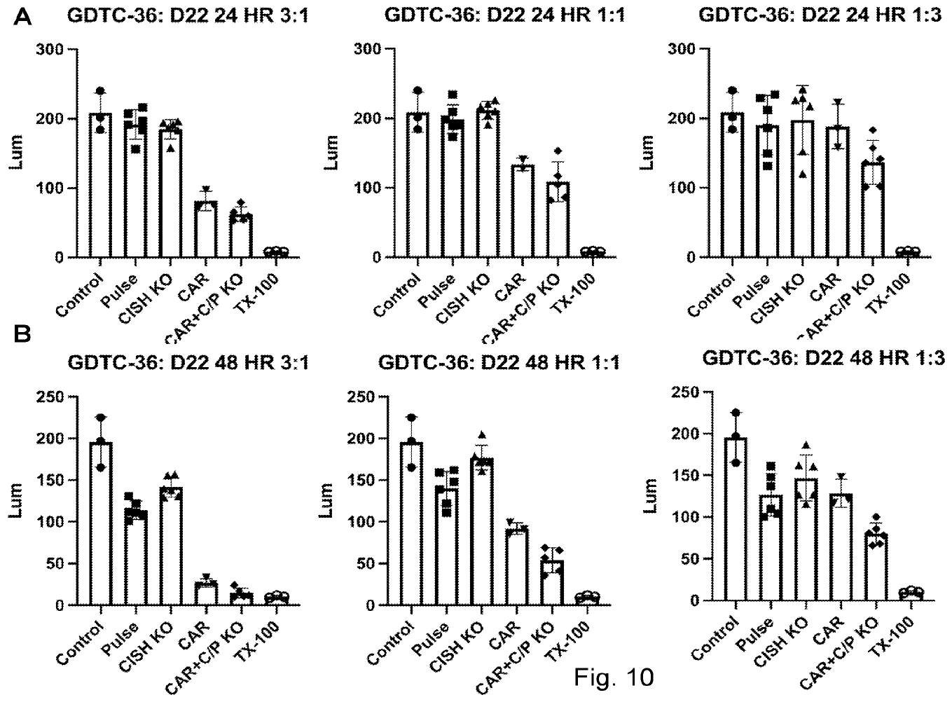

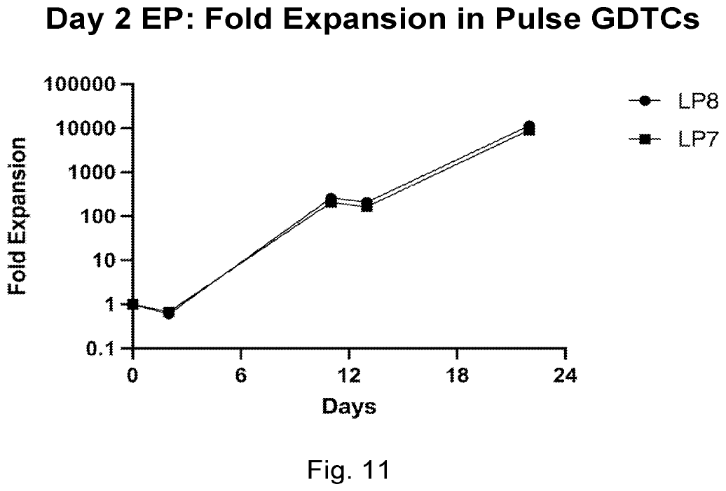

### Day 11 Count Data (FIG. 8A):

Significance: Illustrates total cells for GDTC-33 as a function of electroporation day for the three conditions incorporated on every day: pulse, NP only, and NP+Snoke. The “total cells” graph only takes into account the raw totals, while the “adjusted counts” graph incorporates the expansion that took place between day 2 and the day when each sample (day 4 or day 6) was electroporated.

Notes: Initial density=240k for these graphs. Asterisks indicate a statistically significant difference in day 11 total cells for day 2 electroporations vs day 4/6 electroporations. Values represent multiple comparisons derived from a standard two-way ANOVA. Error bars are one standard deviation above and below the mean. 2 Donors: LP7 and LP8.

Day 11 GFP Expression Data for Nanoplasmid (NP)+Snoke EP cells (FIG. 8B):

Snoke is a hyperactive Tc-Buster transposase that the inventors used to insert the CAR-containing nanoplasmid transposon into the genomes of our target cells. After recognizing transposon terminal repeat sequences within the nanoplasmid, the transposase (Snoke) integrates the CAR construct into the cell genome. Although it yields nonspecific integration, transposons represent a non-viral method of gene transfer, and the inventors have used Snoke to consistently achieve>40% stable integration in gamma delta T cells.

Significance: Represents further validation for choosing day 2, as the cells demonstrated by far the highest CAR integration rate (˜44%) 48 hours post-thaw.

Notes: Asterisks indicate a statistically significant difference in GFP expression for day 2 NP+Snoke EP cells vs day 4/6 EP cells. Values represent multiple comparisons derived from a standard one-way ANOVA. Error bars are one standard deviation above and below the mean

### Day 24 GFP Total Cell Data (FIG. 8C):

Significance: Provides further justification for the use of a D11 re-stim, since unselected NP+Snoke cells that received it grew out significantly more than ones that didn't. Also demonstrates a potential link between no re-stim and crashing out, although this could just be a function of seeding density.

Notes: NS=no re-stim, YS=yes D11 re-stim. Control refers to non-selected NP+Snoke cells, while MTX refers to methotrexate-selected NP+Snoke cells. Error bars are one standard deviation above and below the mean. Asterisks indicate a statistically significant difference in total cells at day 24 between selected and unselected control cells. Values represent multiple comparisons derived from a standard one-way ANOVA.

### Example 2-Production and Functional Characterization of Multiplex, Non-Virally Engineered Gamma Delta T Cells

### GDTC-34:

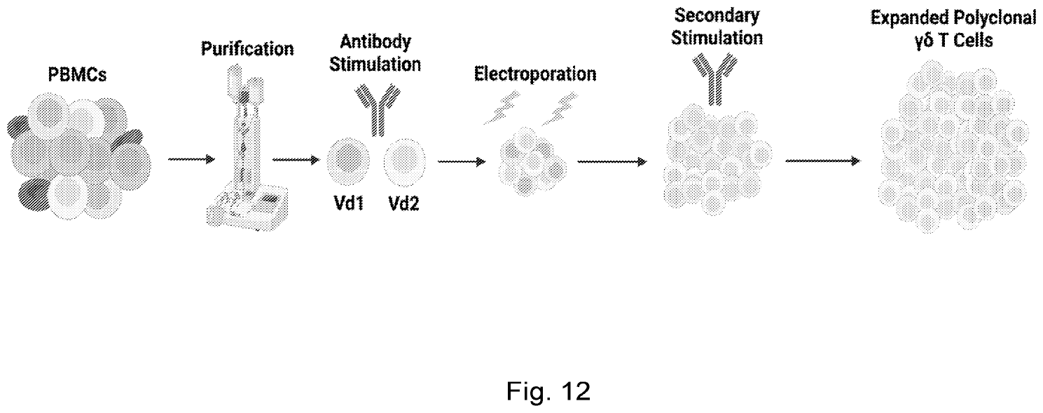

### Representative Protocol for GDTC Engineering (FIG. 12):

Significance: Illustrates optimized expansion protocol for polyclonal GDTCs. GDTCs are purified by negative selection from PBMCs, then stimulated with plate-bound pan-GDTCR Ab+soluble CD28. After 36-48 hours, cells are electroporated with genome engineering reagents, then expanded until day 11. Cells are then re-stimulated with plate-bound pan-GDTCR Ab+ soluble CD28 and expanded until day 22, yielding a polyclonal GDTC population with both Vd1+ and Vd2+ cells.

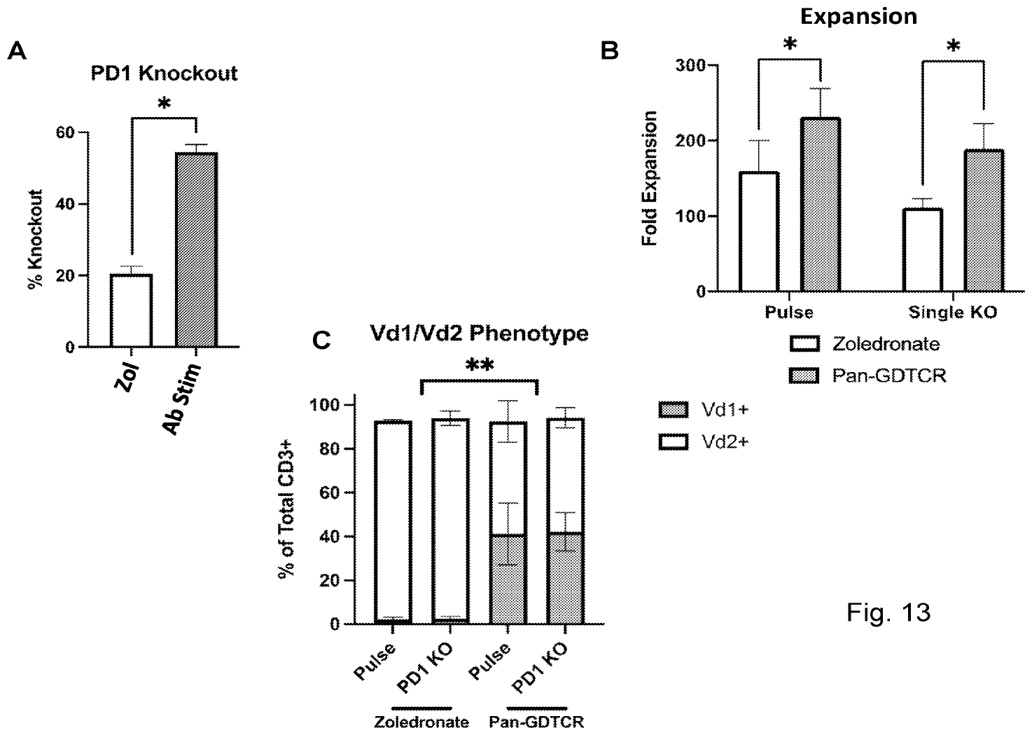

### PD1 Knockout in Day 11 GDTCs (FIG. 13A):

Significance: Demonstrates that pan-GDTCR-stimulated GDTCs, expanded according to the protocol in FIG. 12, are more amenable to CRISPR/Cas9 engineering than zoledronate-stimulated GDTCs. This likely occurs because pan-GDTCR stimulated GDTCs were enriched prior to stimulation, allowing the electroporation of a pure population. Zoledronate stimulation requires feeder cells (PBMCs) to produce phosphoantigens, meaning electroporation must be performed on heterogeneous cell populations.

Notes: The following criteria apply both to this figure (FIG. 13A) and to all following ones (FIG. 13B-26): Asterisks indicate statistical significance (*p<0.05, ** p<0.01). Error bars represent +/−1 standard deviation above and below the mean. Student's T test was used to evaluate significance between two sample means. In cases with >2 populations (i.e. FIG. 13C), one-way ANOVAs were used to evaluate significance.

### Expansion of Day 11 GDTCs (FIG. 13B):

Significance: Illustrates that Pan-GDTCR-stimulated GDTCs expand significantly more following electroporation than zoledronate-stimulated GDTCs.

Notes: “Pulse” indicates cells that were electroporated but did not receive additional engineering reagents. “Single KO” cells received Cas9 mRNA and a PD1 sgRNA.

### Vd1/Vd2 Distribution in D11 GDTCs: (FIG. 13C):

Significance: Demonstrates that the expansion protocol detailed in FIG. 12 leads to the outgrow of polyclonal GDTCs. In both pulse and PD1 KO conditions, day 11 GDTCs exhibit much greater TCR-delta diversity in pan-GDTCR-stimulated populations relative to zoledronate-stimulated ones (which are almost exclusively Vg9Vd2). Polyclonality allows for a greater diversity of functional activities and enhanced therapeutic potential.

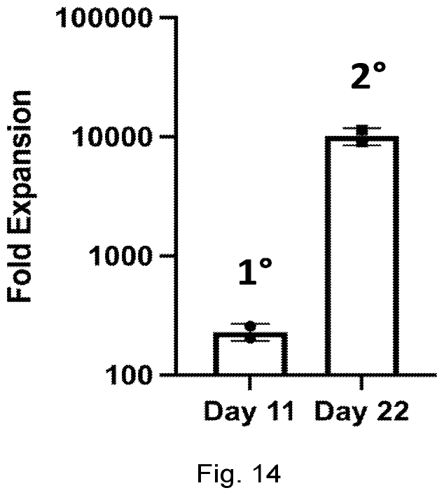

### Fold Expansion Following Primary and Secondary Stimulations (FIG. 14):

Significance: Demonstrates that a re-stimulation significantly enhances expansion of pan-GDTCR-stimulated GDTCs. While pulse cells experience a 200- to 250-fold expansion on day 11, the addition of a re-stimulation followed by 11 more days in culture yields ˜10,000-fold expansion by day 22.

### GDTC-39:

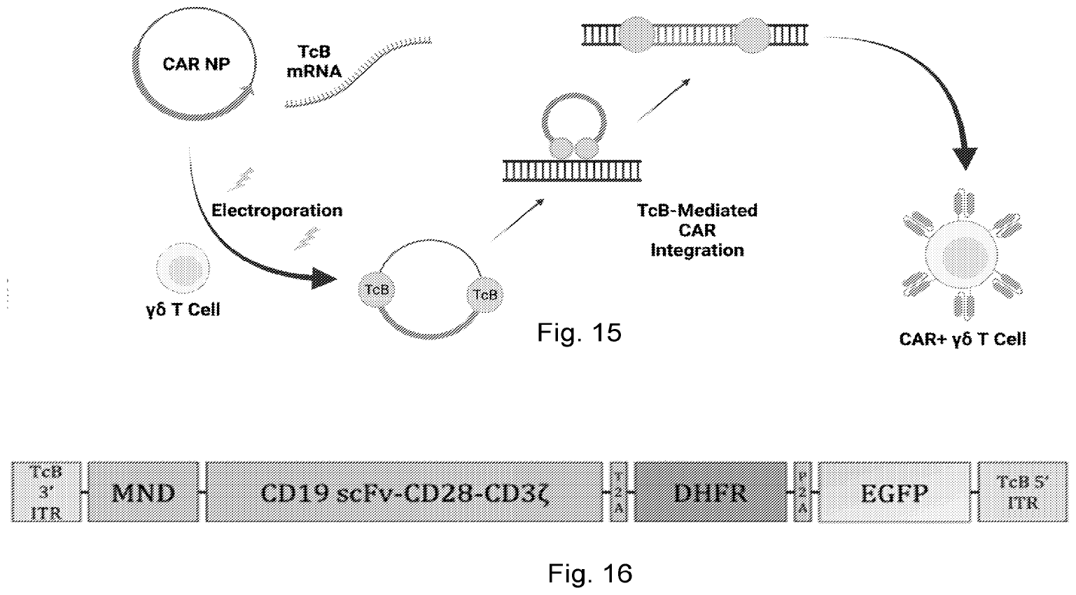

### General Strategy for Transposon-Based Nonviral Genome Engineering (FIG. 15):

Significance: Provides an outline of transposon-based CAR integration in GDTCs. During electroporation, Ab-stimmed GDTCs receive a CAR nanoplasmid (NP) and TcBuster (TcB) transposase mRNA. Once inside the nucleus, TcB mediates nonviral insertion of the CAR DNA sequence into chromosomal DNA. Expression of this sequence produces a CAR protein construct on the outside of the GDTC. This confers enhanced GDTC activation against tumor-associated antigens.

### Representative CAR Construct Design for GDTCs (FIG. 16):

Significance: Provides an illustration of the primary CAR construct used for GDTC engineering. Expression of the construct is driven by a constitutive MND promoter. A CD19 CAR sequence, including an anti-CD19 scFv, CD28 costimulatory domain, and CD3ζ signal transduction domain, is used to drive cytotoxicity against CD19+ cancer cells. EGFP serves as a fluorescence reporter for identification of CAR+ cells by flow cytometry. A mutant DHFR gene is included to confer resistance to methotrexate, allowing for selection of CAR+ cells. P2A and T2A sequences promote independent translation of the CAR, DHFR, and EGFP elements from a single mRNA. TcB binds to 5′ and 3′ flanking sites to mediate integration of the construct into chromosomal DNA.

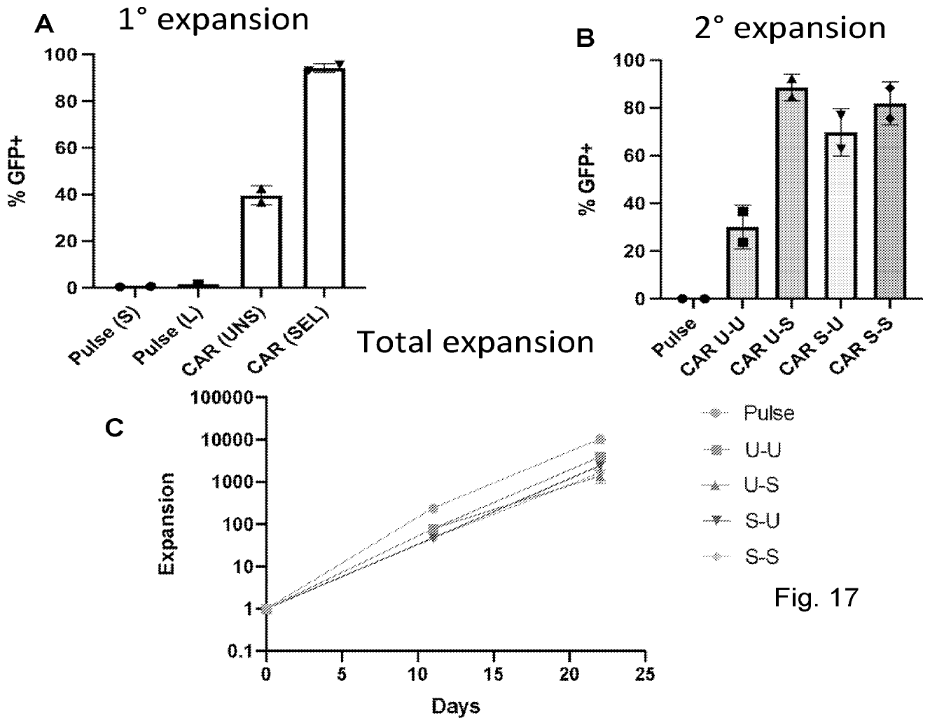

### Selection of Engineered GDTC During Large-Scale Expansion (FIG. 17):

Significance: Demonstrates that engineered cells can be enriched by drug selection. In this instance methotrexate is used to select for GDTC expressing a mutant dihydrofolate reductase (DHFR) gene, which acts as a selectable marker by conferring resistance to methotrexate. Selection can be used during the first and second round of expansion to achieve effective enrichment of engineered GDTC. Selection of engineered GDTC minimally inhibits total cell expansion.

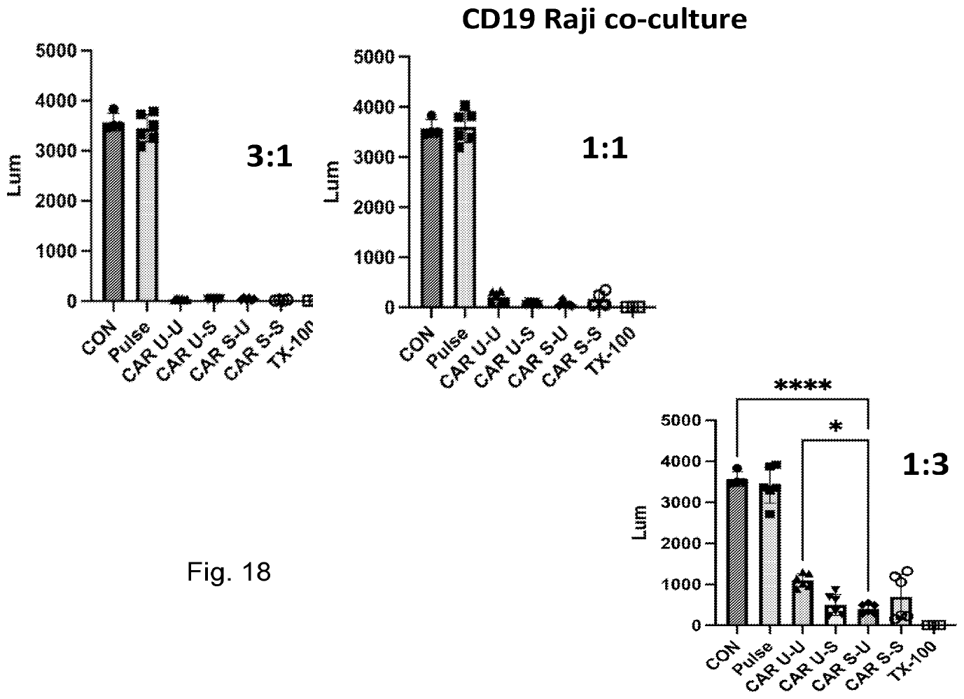

### GDTC Enriched by Methotrexate Selection Retain Cytotoxic Function (FIG. 18):

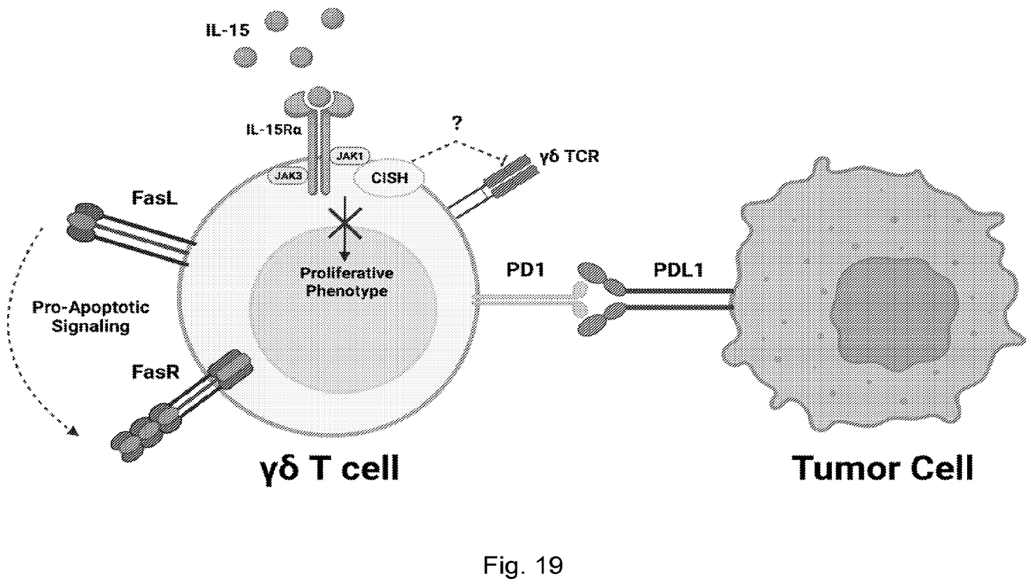

Significance: Transposon engineered GDTC expressing CD19 CAR followed by large scale expansion with or without methotrexate selection mediate effective cytotoxic effects against CD19 expressing Raji cells. Mechanism of GDTC suppression through PD1 by tumor PD-L1 expression (FIG. 19):

Significance: This diagram illustrates a mechanism of GDTC suppression whereby the GDTC expresses PD1 and the tumor expresses PD-L1. This illustrates how disruption of PD1 in engineered GDTC may confer enhanced anti-tumor activity

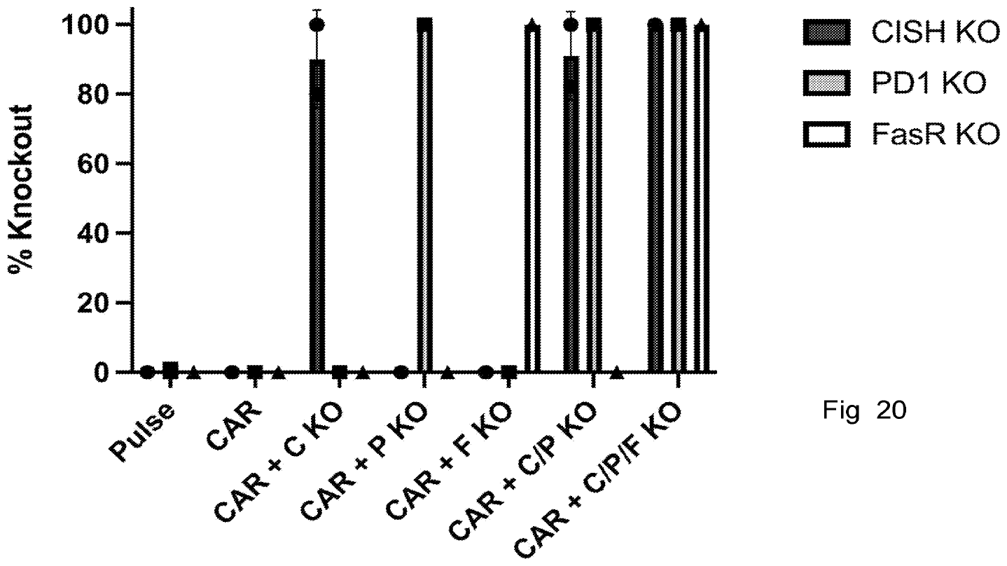

### Highly Efficient, Multiplex Gene Editing by Cas9 Base Editor in GDTC Cells (FIG. 20):

Significance: This data demonstrates that Cas9 base editing, in this case using an adenine base editor (ABE8e), is capable of highly efficient, multiplex gene editing in GDTC during the large-scale expansion protocol. This experimental data demonstrates the knockout of PD1, CISH, and the installation of endogenous dominant negative variant of FAS, all of which are known to restrict T cell function within the tumor microenvironment.

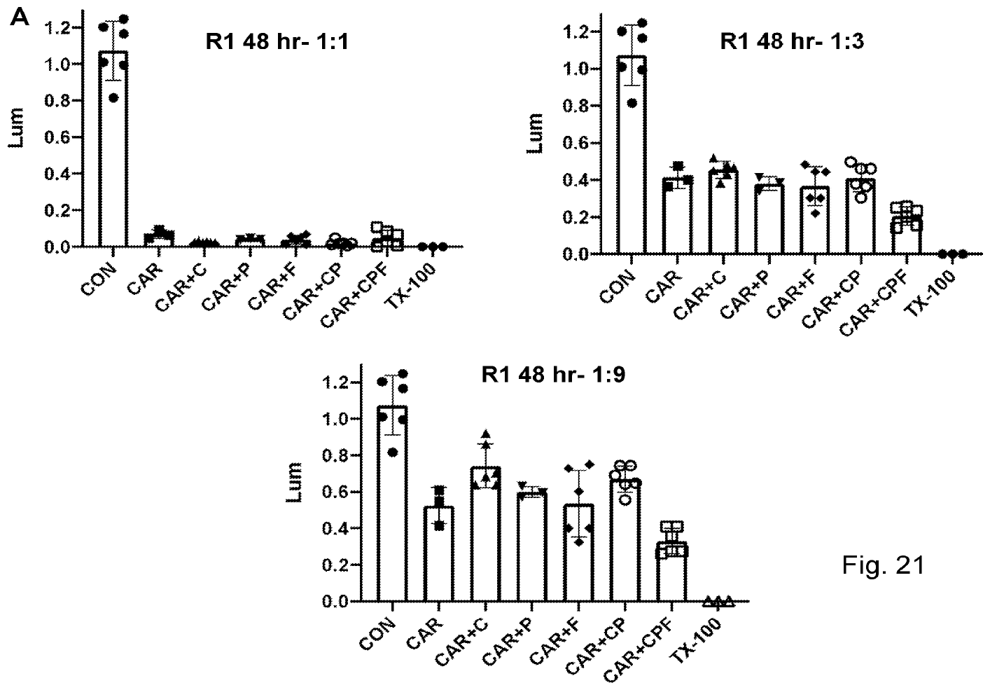

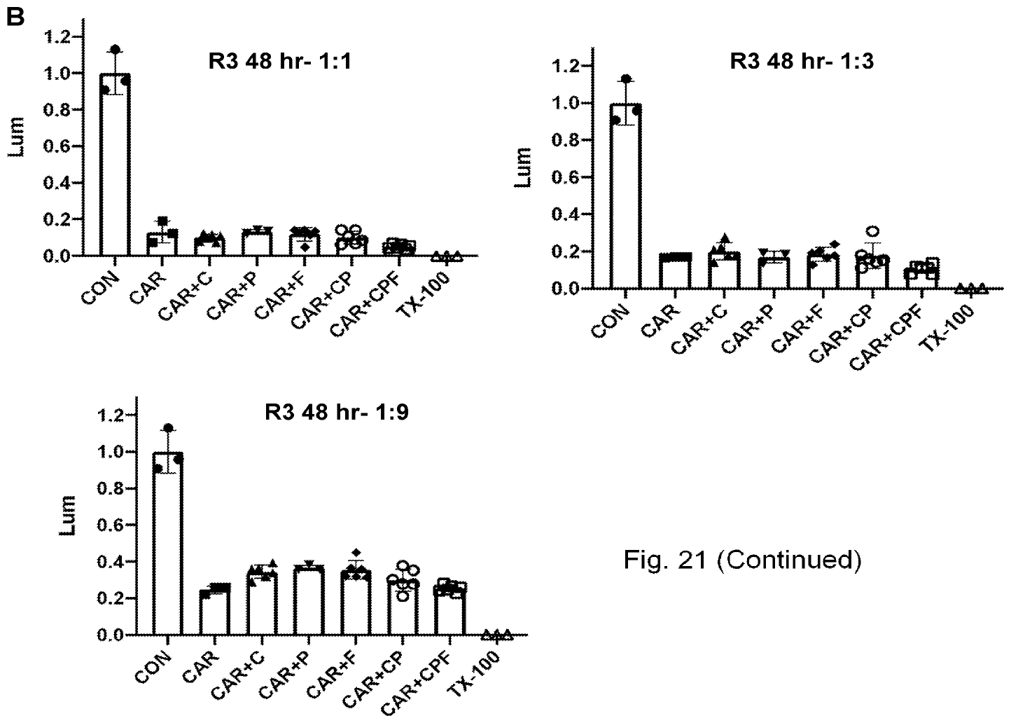

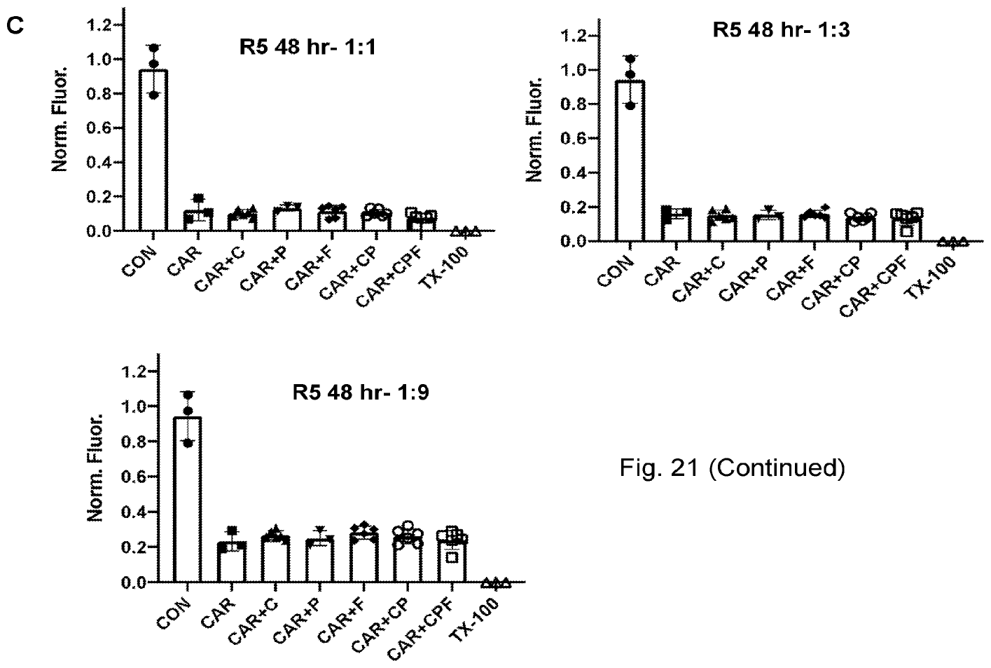

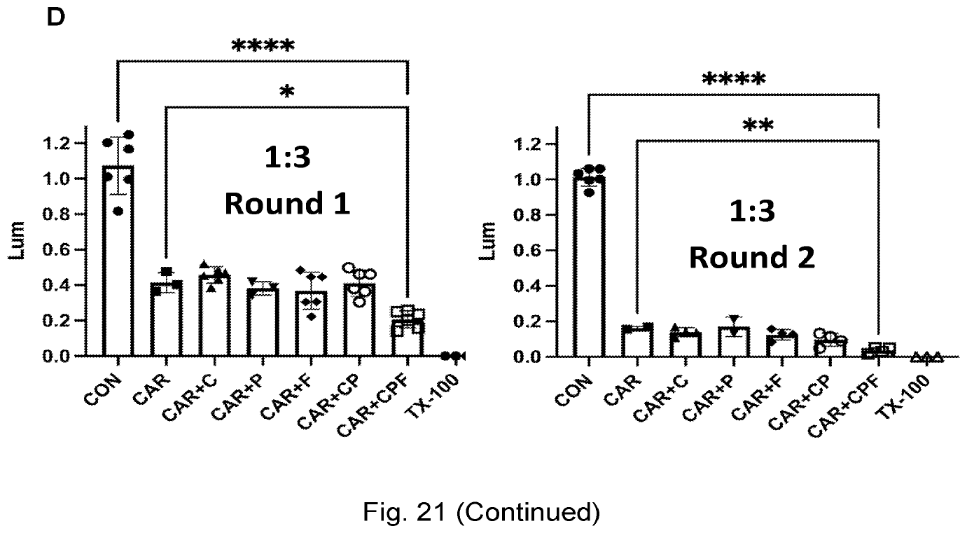

### Cytotoxic Capability of CD19 CAR+ Checkpoint KO GDTC During Serial k (FIG. 21):

Significance: GDTC engineered by transposon to express CD19 CAR while simultaneously edited with ABE8e mRNA and sgRNA to disrupt checkpoint genes CISH, PD1, and FAS alone or in combination elicited effective cytotoxicity against CD19+ Raji target cells following multiple rounds of co-culture. During rounds 1 and 2 at a 1:3 effector to target ratio the CD19 CAR+, CISH/PD1/FAS triple edited GDTC (CAR+CPF) group showed increased cytotoxicity against Raji targets compared to single edited and control/unedited GDTC.

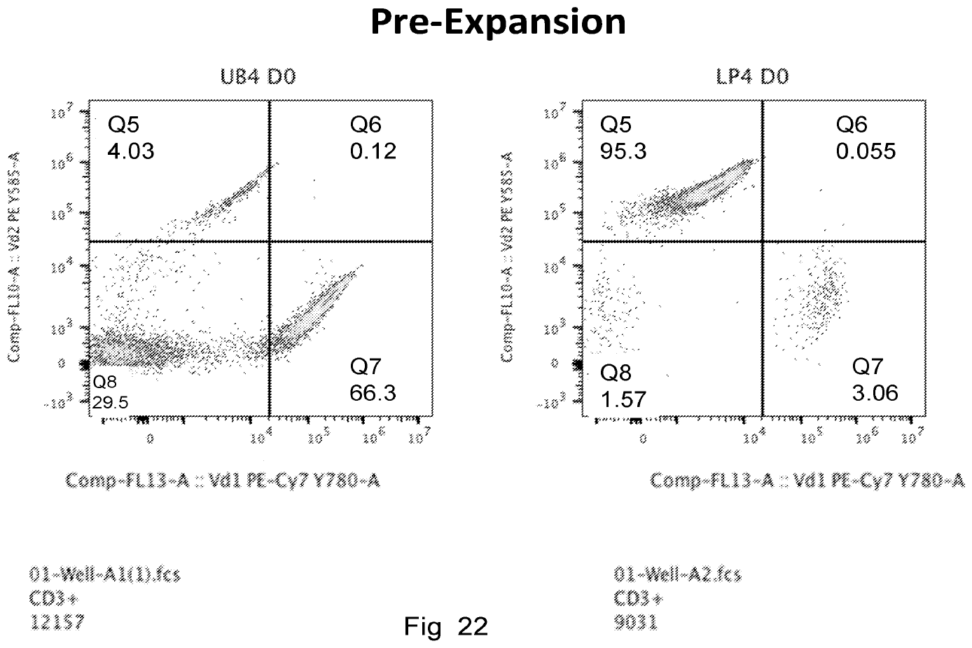

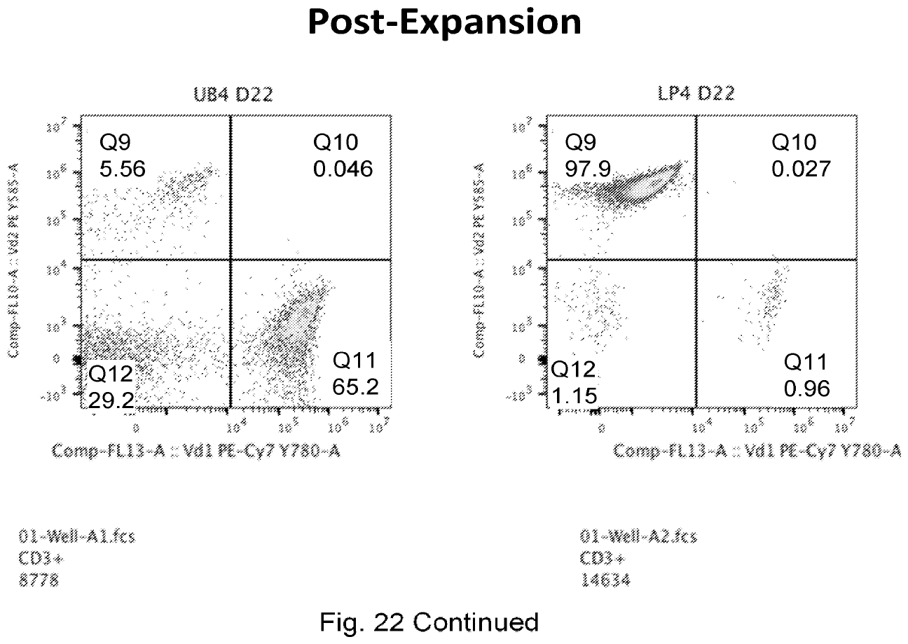

Vd1/Vd2 Distribution in Expanded GDTCs from Adult Peripheral and Umbilical Cord Blood: (FIG. 22):

Significance: Demonstrates that the expansion protocol detailed in FIG. 12 leads to the outgrowth of polyclonal GDTCs from both peripheral blood (LP) and umbilical cord blood (UB). The Vd2+ subset is much more prevalent in peripheral blood samples and this distribution is retained after large scale expansion using the protocol detailed in FIG. 12. The Vd1+ subset is much more prevalent in umbilical cord blood samples and this distribution is retained after large scale expansion using the protocol detailed in FIG. 12.

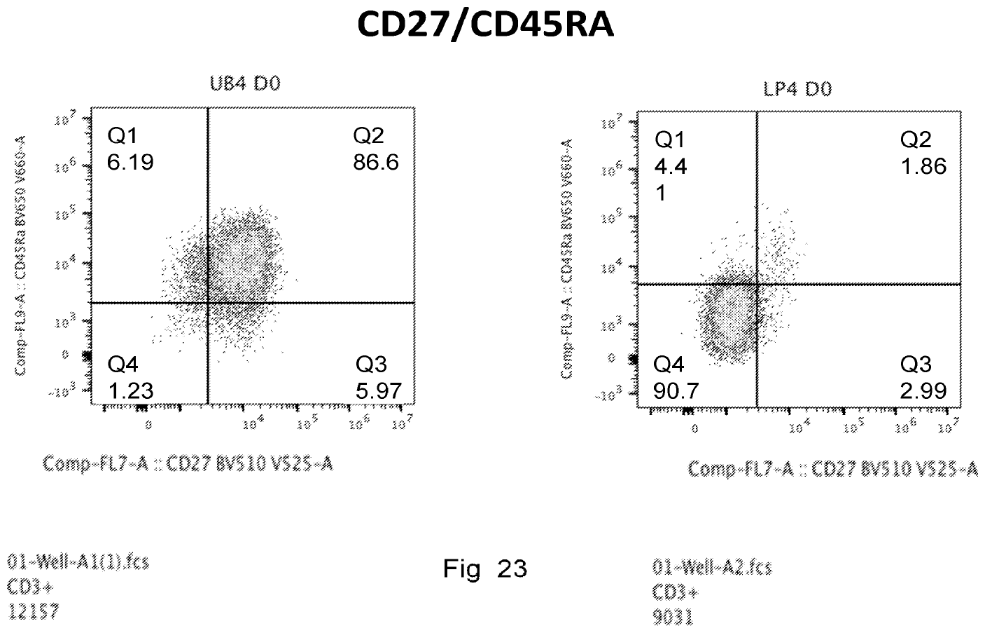

Memory Phenotype of GDTC from Adult Peripheral and Umbilical Cord Blood: (FIG. 23):

Significance: These data illustrate the memory phenotype (CD27/CD45RA) of polyclonal GDTCs from both peripheral blood (LP) and umbilical cord blood (UB). The data indicate that expanded UB GDTC maintain a less differentiated CD45RA+CD27+ phenotype following large scale expansion. Expanded LP GDTC exhibit a CD45RA-CD27− phenotype following large scale expansion suggesting a more differentiated phenotype in adult GDTC.

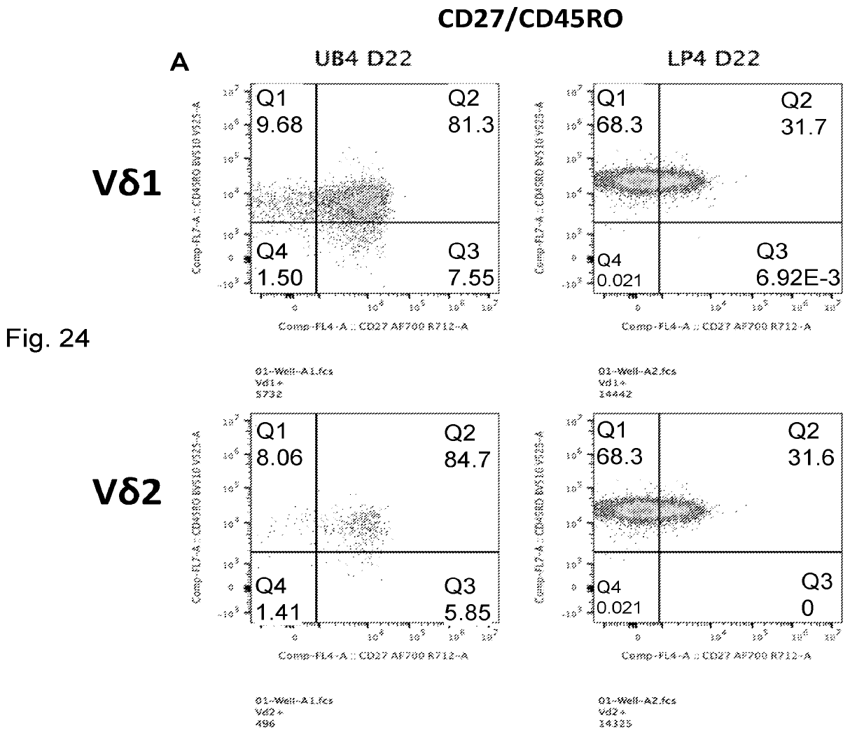

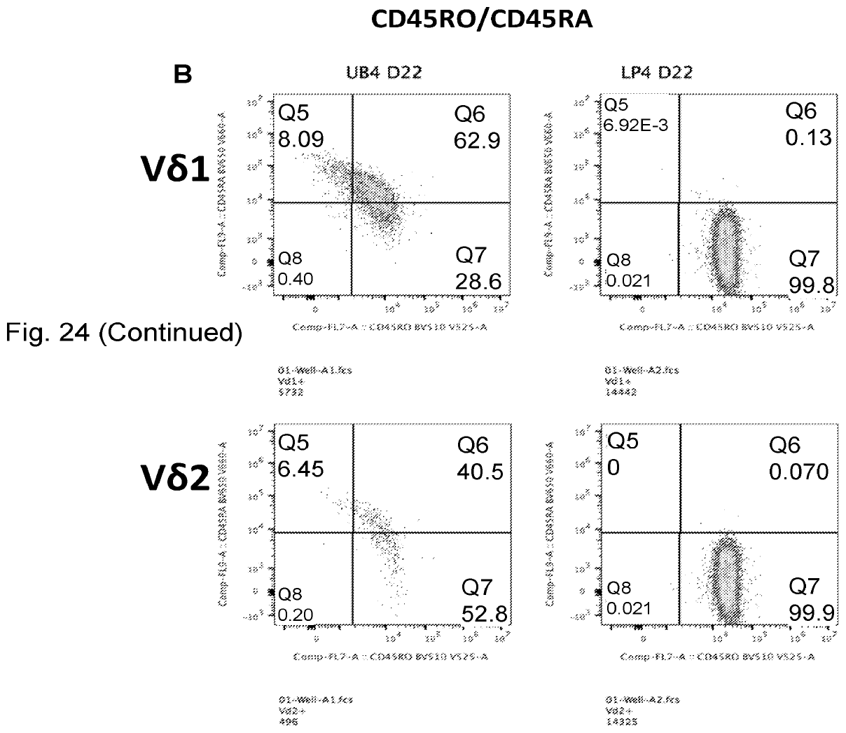

### Differential Expression of Maturity Markers in Cord and Peripheral Blood GDTCs Following Ab-Mediated Expansion (FIG. 24):

Significance: Demonstrates that cord blood GDTCs continue to express naïve-like markers, including CD27 and CD45RA, after 22 days of pan-GDTCR-mediated expansion. However, the cord blood GDTCs also appear to be in a state of maturation, migrating from a CD45RA+/CD45RO-population at day 0 to a CD45RA+/CD45RO+ double positive population at day 22. This is contrasted with peripheral blood cells, which remain predominantly CD27−CD45RA−/CD45RO+ across the duration of the experiment. The mix of naïve and mature phenotypic markers may confer unique therapeutic properties to cord blood GDTCs.

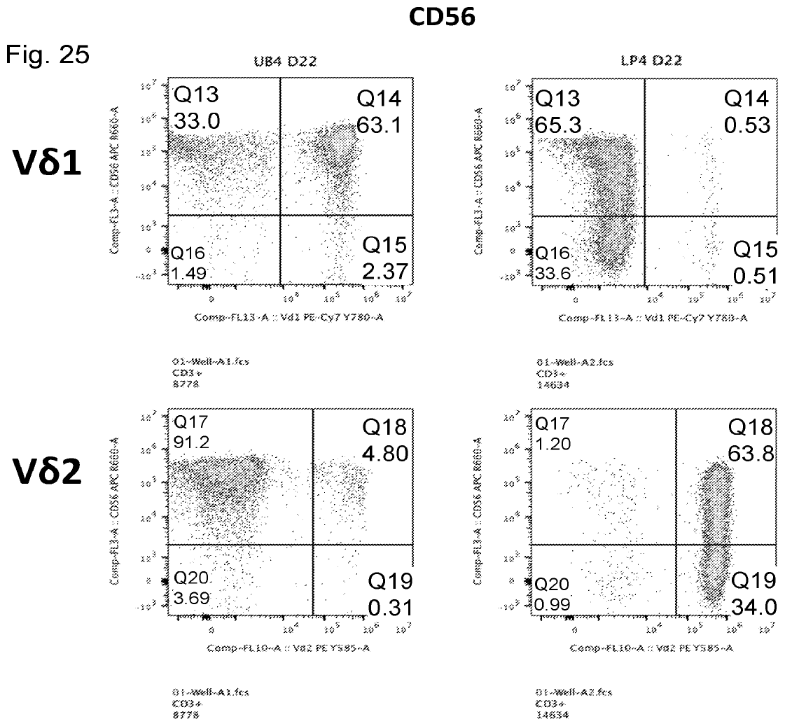

### Differential Expression of Activation Markers in Cord and Peripheral Blood GDTCs Following Ab-Mediated Expansion (FIG. 25):

Significance: Demonstrates that cord blood GDTCs express CD56, a GDTC and NK cell activation marker, at higher rates that peripheral blood GDTCs after 22 days of pan-GDTCR-mediated expansion. This suggests that cord blood GDTCs may respond more robustly to Ab-mediated TCR activation in vitro, potentially yielding more potent functional activity and anti-tumor cytotoxicity.

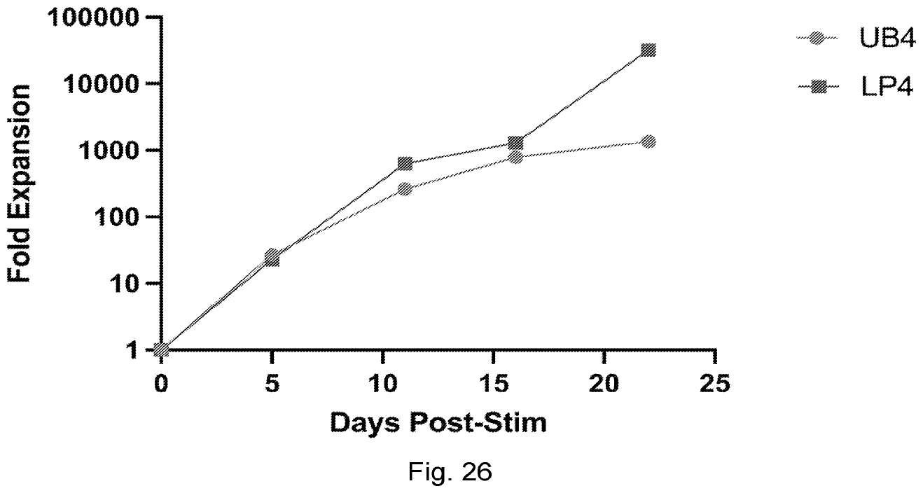

Representative Expansion of Cord Blood and Peripheral Blood GDTCs Stimulated with Pan-GDTCR Ab (FIG. 26):

Significance: Demonstrates robust expansion of both cord blood (UB4) and peripheral blood (LP4) donors in response to pan-GDTCR stimulations at day 0 and day 11. Although LP4 experienced a much higher overall fold expansion than UB4 (>30,000-fold vs 1300-fold), the cord blood cells appeared to expand more rapidly in the first 5 days post-stimulation. We believe that the differences in overall fold expansion were due to the cord blood cells requiring additional stimulation sooner than peripheral blood ones and beginning to die off as a result. Thus, by providing an earlier day 8 or 9 restimulation to the cord blood GDTCs, we believe much greater overall fold expansion can be achieved.

1. A method of expanding polyclonal gammadelta T cells (GDTCs) in vitro comprising:
a) incubating GDTCs in media comprising:
i) anti-gammadelta T cell receptor (GDTCR) antibody;
ii) anti-CD28 antibody; and
iii) one or more of IL-2, IL-7, IL-15 to activate the GDTCs;
b) culturing the GDTCs to expand the GDTCs; and
c) re-stimulating the GDTCs of step (b) by incubating the GDTCs in medium comprising:
i) anti-gammadelta T cell receptor antibody; and
ii) anti-CD28 antibody to re-stimulate the polyclonal GDTCs.

2. The method of claim 1, wherein the GDTCs are not cultured in the presence of a bisphosphonate or in the presence of feeder cells.

3. The method of claim 1, further comprising at step (b) genetically engineering the cells to alter the expression of one or more proteins in the GDTCs.

4. The method of claim 3, wherein the cells are genetically engineered using a CRISPR/Cas system.

5. The method of claim 4, wherein a Cas nuclease and one or more guide RNAs are introduced into the cells to allow for genetic engineering.

6. The method of claim 5, wherein the guide RNAs are specific for at least one of cytokine inducible SH2 containing protein (CISH), programmed death-1 (PD-1), and Fas receptor (FasR) and result in reduced activity of the protein.

7-10. (canceled)

11. The method of claim 1, wherein the cells are genetically engineered by contacting the cells with a vector encoding a transposon.

12. (canceled)

13. The method of claim 1, wherein the population of polyclonal GDTCs are expanded at least 100-fold.

14-16. (canceled)

17. The method of claim 1, wherein the anti-gammadelta T cell receptor antibody is linked to a solid support.

18. The method of claim 17, wherein the solid support comprises the surface of the vessel in which the GDTCs are cultured in step b).

19. The method of claim 17, wherein the solid support comprises beads.

20. The method of claim 1, wherein the method further comprises contacting the cells with a FasL blocking reagent.

21. The method of claim 1, wherein step (a) to activate the GDTCs is about 1-3 days.

22. The method of claim 1, wherein step (c) to re-stimulate the GDTCs is about 1-3 days.

23. (canceled)

24. The method of claim 1, wherein the method produces a population of gammadelta T cells (GDTCs).

25. A kit for expanding polyclonal gammadelta T cells (GDTCs) comprising:
i) anti-gammadelta T cell receptor (GDTCR) antibody;
ii) anti-CD28 antibody; and
iii) one or more, two or more, or all three of IL 2, IL-7, IL-15 cytokines.

26-28. (canceled)

29. The kit of claim 25, further comprising:
iv) a FasL blocking reagent.

30. A method of treating a cell proliferative disease or disorder in a subject in need thereof, the method comprising: administering the population of gammadelta T cell of claim 24 to a subject in need thereof to treat the cell proliferative disease or disorder.

31. The method of claim 30, wherein the cell proliferative disease or disorder is selected from: bone cancer, brain cancer, breast cancer, cervical cancer, cancer of the larynx, lung cancer, pancreatic cancer, prostate cancer, skin cancer, cancer of the spine, stomach cancer, uterine cancer, hematopoietic cancer, and/or lymphoid cancer.

32. The method of claim 31, wherein the cell proliferative disease or disorder comprises: acute myelogenous leukemia (AML), acute lymphoblastic leukemia (ALL), myelodysplastic syndromes (MDS), non-Hodgkin lymphoma (NHL), chronic myelogenous leukemia (CML), Hodgkin's disease, or multiple myeloma.

33. A population of expanded genetically modified polyclonal gammadelta T cells (GDTCs), wherein the GDTCs comprise at least 10 distinct gammadelta T cell receptor (GDTCR) clones.

34.-41. (canceled)
